(19) United States
(12) Patent Application Publication
(10) Pub. No.: US 2026/0263331 A1
(43) Pub. Date: Sep. 10, 2026

(54) HYBRID MICROCAPSULES

(71) Applicant: Firmenich SA, Satigny (CH)

(72) Inventors: Yongtao WU, Shanghai (CN); Jia-Jun SHEN, Shanghai (CN); Shi-Wen GAO, Shanghai (CN); Xuhong GUO, Shanghai (CN)

(21) Appl. No.: 18/875,559

(22) PCT Filed: Jun. 30, 2023
(86) PCT No.: PCT/EP23/68046

§ 371 Date: Dec. 16, 2024

(30) Foreign Application Priority Data
Jul. 21, 2022 (EP) 22186252.7

(51) Int. Cl. A61K 8/11; A01N 25/28; A01P 17/00; A61K 9/50; A61Q 19/00; C11D 17/00

(52) U.S. Cl. CPC A61K 8/11; A01N 25/28; A01P 17/00; A61K 9/5089; A61Q 19/00; C11D 17/0039; A61K 2800/10

(57) ABSTRACT

Disclosed herein is a process for the preparation of hybrid microcapsules. Also disclosed herein are the hybrid microcapsules. Also disclosed herein are perfuming compositions and consumer products including the microcapsules, including perfumed consumer products in the form of home care or personal care products.

### TECHNICAL FIELD

The present invention relates to a new process for the preparation of hybrid microcapsules. Hybrid microcapsules are also an object of the invention. Perfuming compositions and consumer products comprising said microcapsules, in particular perfumed consumer products in the form of home care or personal care products, are also part of the invention.

### BACKGROUND OF THE INVENTION

One of the problems faced by the perfumery industry lies in the relatively rapid loss of olfactive benefit provided by odoriferous compounds due to their volatility, particularly that of “top-notes”. In order to tailor the release rates of volatiles, delivery systems such as microcapsules containing a perfume are needed to protect and later release the core payload when triggered. A key requirement from the industry regarding these systems is to survive suspension in challenging bases without physically dissociating or degrading. This is referred to as stability for the delivery system. For instance, fragranced personal and household cleansers containing high levels of aggressive surfactant detergents are very challenging for the stability of microcapsules.

In addition to the performance in terms of stability and olfactive performance, the consumer demand for eco-friendly delivery systems is more and more important and is driving the development of new delivery systems.

There is therefore still a need to provide new microcapsules using more eco-friendly materials, while not compromising on the performance of the microcapsules, in particular in terms of stability in a challenging medium such as a consumer product base, as well as in delivering a good performance in terms of active ingredient delivery, e.g. olfactive performance in the case of perfuming ingredients.

The present invention is proposing a solution to the above-mentioned problem by providing new hybrid microcapsules and a process for preparing said microcapsules.

### SUMMARY OF THE INVENTION

A first object of the invention is a process for the preparation of a microcapsule slurry comprising the steps of:
1) suspending coacervate particles in a solvent to form a dispersing phase;
2) admixing at least one polyfunctional monomer with a hydrophobic material to form an oil phase;
3) adding the oil phase to the dispersing phase and mixing them to form a two-phases Pickering emulsion under conditions allowing the formation of a microcapsule slurry by interfacial polymerization.

A second object of the invention is a microcapsule slurry obtainable by the process according to anyone of the preceding claims.

The present invention also relates to perfumed consumer products and flavoured edible products comprising the microcapsule slurry or microcapsules defined above.

### BRIEF DESCRIPTION OF THE FIGURES

FIG. 1: is a schematic representation of the formation of a Pickering emulsion when coacervate particles are used to stabilize the oil phase.

FIG. 2: represents microscopic picture of microcapsules prepared by the process of the invention.

FIG. 3: represents the preparation of coacervate particles by using flash nanoprecipitation process (FNP) with two polymer solutions (having opposite charge).

### DETAILED DESCRIPTION OF THE INVENTION

Unless stated otherwise, percentages (%) are meant to designate a percentage by weight of a composition.

By “active ingredient”, it is meant a single compound or a combination of ingredients.

By “perfume or flavour oil”, it is meant a single perfuming or flavouring compound or a mixture of several perfuming or flavouring compounds.

By “consumer product” or “end-product” it is meant a manufactured product ready to be distributed, sold and used by a consumer.

For the sake of clarity, by the expression “dispersion” in the present invention it is meant a system in which particles are dispersed in a continuous phase of a different composition and it specifically includes a suspension or an emulsion.

A “microcapsule”, or the similar, in the present invention it is meant that core-shell microcapsules have a particle size distribution in the micron range (e.g. a mean diameter comprised between about 1 and 3000 microns, preferably between 1 and 500 microns, more preferably between 5 and 50 microns), and comprise an external solid polymeric shell and an internal continuous oil phase enclosed by the external shell.

By “microcapsules size” or “particle size” it is meant the volume mean diameter (D[4,3]) of the relevant capsules/particle, capsules/particles suspension as obtained by laser light scattering of a diluted sample in a Malvern Mastersizer 3000.

The microcapsules are thus defined as “hybrid” or “composite” with reference to the nature of the shell that is composed a polymeric shell formed from the interfacial polymerization, wherein coacervates particles are embedded within said polymeric shell. Coacervates particles are used to stabilize a Pickering emulsion during the process.

By “microcapsule slurry”, it is meant microcapsule(s) that is (are) dispersed in a liquid. According to an embodiment, the slurry is an aqueous slurry, i.e the microcapsule(s) is (are) dispersed in an aqueous phase.

By “polyfunctional monomer”, it is meant a molecule that, as unit, reacts or binds chemically to form a polymer or a supramolecular polymer. The polyfunctional monomer of the invention has at least two functional groups that are capable to react with or bind to functional groups of another component and/or are capable to polymerize to form a polymeric shell.

It has been found that hybrid microcapsules with overall good performance in challenging bases could be obtained.

### Process for Preparing Microcapsules

A first object of the invention is a process for the preparation of a microcapsule slurry comprising the steps of:
1) suspending coacervate particles in a solvent to form a dispersing phase;
2) admixing at least one polyfunctional monomer with a hydrophobic material to form an oil phase;
3) adding the oil phase to the dispersing phase and mixing them to form a two-phases Pickering emulsion under conditions allowing the formation of a microcapsule slurry by interfacial polymerization.
   According to an embodiment, the process for the preparation of a microcapsule slurry comprises the steps of:
1) suspending coacervate particles in water to form a water phase;
2) admixing at least one polyfunctional monomer with a hydrophobic material to form an oil phase;
3) adding the oil phase to the water phase and mixing them to form an oil-in-water Pickering emulsion under conditions allowing the formation of a microcapsule slurry by interfacial polymerization.
   According to an embodiment, the solvent comprises, preferably consists of water.
   According to an embodiment, the dispersing phase is a water phase.
   According to an embodiment, the two-phases dispersion is an oil-in-water emulsion.
   According to an embodiment, the solvent comprises water and an alcohol such as glycerol, 1,4-butanediol, ethylene glycol and mixtures thereof.
   According to an embodiment, a second polyfunctional monomer is added in step 1) in the water phase and/or in step 3) in the Pickering emulsion.
   The second polyfunctional monomer can be chosen in the group consisting of melamine-based resin or urea-based resin.
   According to an embodiment, two processes are combined in the present invention, namely, the formation of coacervate particles prepared by Flash Nanoprecipitation (FNP) process, and an interfacial polymerization (or interfacial reaction) process to obtain microcapsules having good properties.
   The process of the invention comprises forming a Pickering emulsion that is further subjected to interfacial polymerization (or interfacial reaction).

Without wishing to be bound by theory, it is believed that the Pickering emulsion determines the morphology and surface properties (size, density, zeta potential, stiffness) of the membrane, while the interfacial reaction determines the permeability and stiffness of the capsules.

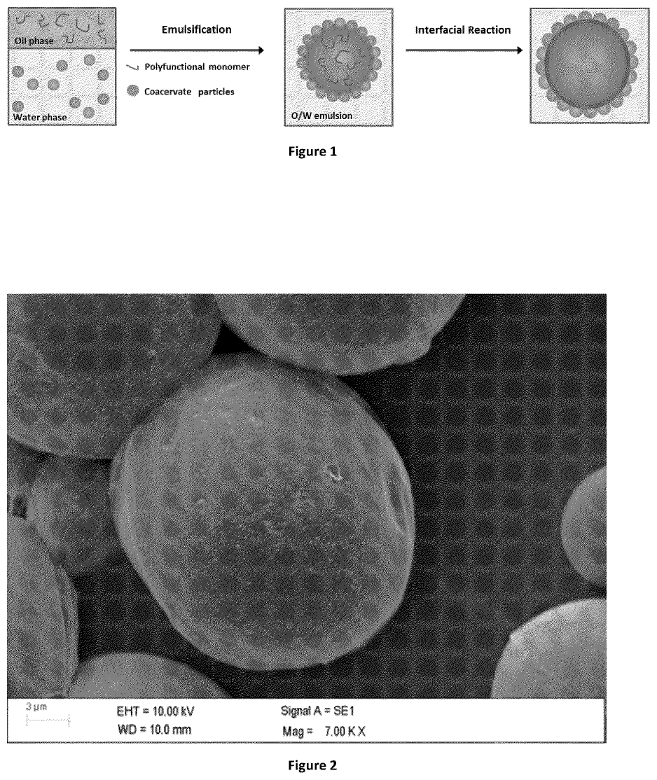

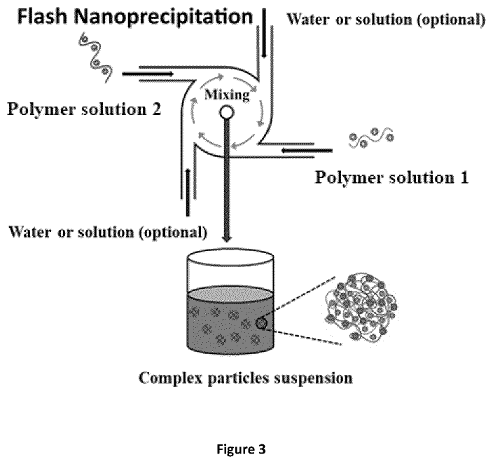

FIG. 1 represents the formation of a Pickering emulsion when coacervate particles are used as colloidal stabilizer to stabilize the oil droplets.

According to a particular embodiment, a polymeric emulsifier is added in step 1) in the water phase.

By “polymeric emulsifier”, it meant an emulsifier having both a polar group with an affinity for water (hydrophilic) and a nonpolar group with an affinity for oil (lipophilic). The hydrophilic part can dissolve in the water phase and the hydrophobic part can dissolve in the oil phase providing a film around droplets. Coacervate particles used in the present invention are not a polymeric emulsifier. Coacervate particles belong to a colloidal stabilizer.

This optional polymeric emulsifier can allow assisting to stabilize the oil droplets in the presence of coacervate particles. The polymeric emulsifier can be an ionic or non-ionic surfactant. As non-limiting examples, non-ionic polymers include polyvinyl alcohol, gum Arabic, cellulose derivatives such hydroxyethyl cellulose, polyethylene oxide, co-polymers of polyethylene oxide and polyethylene or polypropylene oxide, co-polymers alkyl acrylates and N-vinylpyrrolidone, and non-ionic polysaccharide. Ionic polymers include co-polymers of acrylamide and acrylic acid, acid anionic surfactant (such as sodium dodecyl sulfate), acrylic co-polymers bearing a sulfonate group, and co-polymers of vinyl ethers and maleic anhydride, and ionic polysaccharide.

According to an embodiment, no polymeric emulsifier is added at any stage of the process.

In the first step of the process, the coacervate particles are dispersed in a solvent, preferably a water phase, with a pH preferably between 2 and 8. Typically, this is done under ultrasonic or high mechanical agitation condition. In a second step, at least one polyfunctional monomer is dissolved in a hydrophobic material (for example, a perfume or flavour oil) to form an oil phase, which is then added to the dispersing phase (typically water phase) to form a Pickering emulsion, the mean droplet size of which is preferably comprised between 1 and 3000 microns, more preferably between 1 and 500 microns, even more preferably between 5 and 50 microns. The two-phases Pickering dispersion (typically oil-in-water Pickering emulsion) is made for instance by using high speed mechanical disperser or ultrasonic dispersers at room temperature.

Once the Pickering emulsion is formed, the pH value is preferably adjusted to a value above 8.5 and preferably not higher than 11. However, this step can be omitted.

The interfacial polymerization can be carried out typically at a temperature between 50° C. and 80° C. under stirring for 2 to 40 hours to complete the reaction and form microcapsules in the form of a slurry.

The morphology of the microcapsules of the invention can vary from a core-shell to a matrix type. According to one embodiment, it is of the core-shell type. In this case, the microcapsules comprise a core based on a hydrophobic active ingredient, typically a perfume or flavour oil, and a polymeric shell comprising coacervate particles embedded within the polymeric shell.

### Oil Phase

According to the invention, at least one polyfunctional monomer is admixed with a hydrophobic material to form an oil phase.

According to an embodiment, the polyfunctional monomer used in the process of the invention is present in amounts representing from 0.1 to 40%, preferably from 0.1 to 30%, more preferably from 0.2 to 20% by weight based on the total amount of the oil phase.

According to an embodiment, the polyfunctional monomer is chosen in the group consisting of at least one isocyanate, anhydride or maleic anhydride, acyl chloride, epoxide, (meth) acrylate monomers, alkoxysilane, and mixtures thereof.

Suitable polyfunctional isocyanates used according to the invention have at least two or three isocyanate functional groups and include aromatic isocyanate, aliphatic isocyanate and mixtures thereof.

According to a particular embodiment, the polyfunctional isocyanate comprises at least 3 but may comprise up to 6, or even only 4, isocyanate functional groups and the oil phase is essentially free from diisocyanate.

According to a particular embodiment, a triisocyanate (3 isocyanate functional groups) is used.

According to one embodiment, said polyfunctional isocyanate is an aromatic polyfunctional isocyanate.

The term “aromatic polyfunctional isocyanate” is meant here as encompassing any polyfunctional isocyanate comprising an aromatic moiety. Preferably, it comprises a phenyl, a toluyl, a xylyl, a naphthyl or a diphenyl moiety, more preferably a toluyl or a xylyl moiety. Preferred aromatic polyfunctional isocyanates are biurets and polyisocyanurates, more preferably comprising one of the above-cited specific aromatic moieties. More preferably, the aromatic polyfunctional isocyanate is a polyisocyanurate of toluene diisocyanate (commercially available from Bayer under the tradename Desmodur® RC), a trimethylol propane-adduct of toluene diisocyanate (commercially available from Bayer under the tradename Desmodur® L75), a trimethylol propane-adduct of xylylene diisocyanate (commercially available from Mitsui Chemicals under the tradename Takenate® D-110N). In a most preferred embodiment, the aromatic polyfunctional isocyanate is a trimethylol propane-adduct of xylylene diisocyanate.

According to another embodiment, said polyfunctional isocyanate is an aliphatic polyfunctional isocyanate. The term “aliphatic polyfunctional isocyanate” is defined as a polyfunctional isocyanate which does not comprise any aromatic moiety. Preferred aliphatic polyfunctional isocyanates are a trimer of hexamethylene diisocyanate, a trimer of isophorone diisocyanate, a trimethylol propane-adduct of hexamethylene diisocyanate (available from Mitsui Chemicals) or a biuret of hexamethylene diisocyanate (commercially available from Bayer under the tradename Desmodur® N 100), among which a biuret of hexamethylene diisocyanate is even more preferred.

According to another embodiment, said at least one polyfunctional isocyanate is in the form of a mixture of at least one aliphatic polyfunctional isocyanate and of at least one aromatic polyfunctional isocyanate, both comprising at least two or three isocyanate functional groups, such as a mixture of a biuret of hexamethylene diisocyanate with a trimethylol propane-adduct of xylylene diisocyanate, a mixture of a biuret of hexamethylene diisocyanate with a polyisocyanurate of toluene diisocyanate and a mixture of a biuret of hexamethylene diisocyanate with a trimethylol propane-adduct of toluene diisocyanate. Most preferably, it is a mixture of a biuret of hexamethylene diisocyanate with a trimethylol propane-adduct of xylylene diisocyanate.

According to an embodiment, the polyfunctional monomer is an acyl chloride.

According to a particular embodiment, the acyl chloride has the following formula (I)

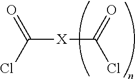

wherein n is an integer varying between 1 and 8, preferably between 1 and 6, more preferably between 1 and 4, and
wherein X is an (n+1)-valent C₂ to C₄₅ hydrocarbon group optionally comprising at least one group selected from (i) to (xi), particularly from (i) to (vi)

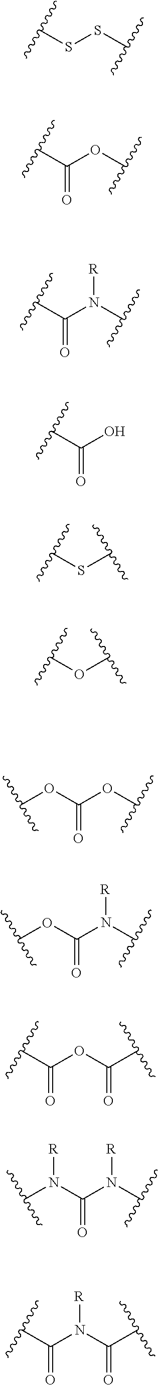

wherein R is a hydrogen atom or an alkyl group such as a methyl or an ethyl group, preferably a hydrogen atom.

It is understood that by “ . . . hydrocarbon group . . . ” it is meant that said group consists of hydrogen and carbon atoms and can be in the form of an aliphatic hydrocarbon, i.e. linear or branched saturated hydrocarbon (e.g. alkyl group), a linear or branched unsaturated hydrocarbon (e.g. alkenyl or alkynil group), a saturated cyclic hydrocarbon (e.g. cycloalkyl) or an unsaturated cyclic hydrocarbon (e.g. cycloalkenyl or cycloalkynyl), or can be in the form of an aromatic hydrocarbon, i.e. aryl group, or can also be in the form of a mixture of said type of groups, e.g. a specific group may comprise a linear alkyl, a branched alkenyl (e.g. having one or more carbon-carbon double bonds), a (poly)cycloalkyl and an aryl moiety, unless a specific limitation to only one type is mentioned. Similarly, in all the embodiments of the invention, when a group is mentioned as being in the form of more than one type of topology (e.g. linear, cyclic or branched) and/or being saturated or unsaturated (e.g. alkyl, aromatic or alkenyl), it is also meant a group which may comprise moieties having any one of said topologies or being saturated or unsaturated, as explained above. Similarly, in all the embodiments of the invention, when a group is mentioned as being in the form of one type of saturation or unsaturation, (e.g. alkyl), it is meant that said group can be in any type of topology (e.g. linear, cyclic or branched) or having several moieties with various topologies.

It is understood that with the term “ . . . a hydrocarbon group, optionally comprising . . . ” it is meant that said hydrocarbon group optionally comprises heteroatoms to form ether, thioether, amine, nitrile or carboxylic acid groups and derivatives (including for example esters, acids, amide). These groups can either substitute a hydrogen atom of the hydrocarbon group and thus be laterally attached to said hydrocarbon, or substitute a carbon atom (if chemically possible) of the hydrocarbon group and thus be inserted into the hydrocarbon chain or ring.

According to a particular embodiment, the acyl chloride is chosen from the group consisting of benzene-1,3,5-tricarbonyl trichloride (trimesoyl trichloride), benzene-1,2,4-tricarbonyl trichloride, benzene-1,2,4,5-tetracarbonyl tetrachloride, cyclohexane-1,3,5-tricarbonyl trichloride, isophthalyol dichloride, diglycolyl dichloride, terephthaloyl chloride, fumaryl dichloride, adipoyl chloride, succinic dichloride, propane-1,2,3-tricarbonyl trichloride, cyclohexane-1,2,4,5-tetracarbonyl tetrachloride, 2,2′-disulfanediyldisuccinyl dichloride, 2-(2-chloro-2-oxo-ethyl)sulfanylbutanedioyl dichloride, (4-chloro-4-oxobutanoyl)-L-glutamoyl dichloride, (S)-4-((1,5-dichloro-1,5-dioxopentan-2-yl)amino)-4-oxobutanoic acid, 2,2-bis[(4-chloro-4-oxo-butanoyl)oxymethyl]butyl 4-chloro-4-oxo-butanoate, [2-[2,2-bis[(4-chloro-4-oxo-butanoyl)oxymethyl]butoxymethyl]-2-[(4-chloro-4-oxo-butanoyl)oxymethyl]butyl]4-chloro-4-oxo-butanoate, 2,2-bis[(2-chlorocarbonylbenzoyl)oxymethyl]butyl 2-chlorocarbonyl-benzoate, [2-[2,2-bis[(2-chlorocarbonylbenzoyl)oxymethyl]butoxymethyl]-2-[(2-chlorocarbonylbenzoyl)oxymethyl]butyl] 2-chlorocarbonylbenzoate, 4-(2,4,5-trichlorocarbonylbenzoyl)oxybutyl 2,4,5-trichlorocarbonyl-benzoate, propane-1,2,3-triyl tris(4-chloro-4-oxobutanoate), propane-1,2-diyl bis(4-chloro-4-oxobutanoate) and mixtures thereof.

According to a particular embodiment, the oil phase concentration is comprised between 5% and 60%, preferably between 20% and 40% of the Pickering emulsion.

### Hydrophobic Material

According to an embodiment, the core is an oil-based core.

The hydrophobic material according to the invention can be “inert” material like solvents or active ingredients.

When the hydrophobic materials is an active ingredient, it is preferably chosen from the group consisting of flavors, flavor ingredients, perfumes, perfume ingredients, nutraceuticals, cosmetics, pest control agents, biocide actives and mixtures thereof.

According to a particular embodiment, the hydrophobic material comprises a mixture of a perfume with another ingredient selected from the group consisting of nutraceuticals, cosmetics, pest control agents and biocide actives.

According to an embodiment, the hydrophobic material comprises a phase change material (PCM).

According to a particular embodiment, the hydrophobic material comprises a mixture of biocide actives with another ingredient selected from the group consisting of perfumes, nutraceuticals, cosmetics, pest control agents.

According to a particular embodiment, the hydrophobic material comprises a mixture of pest control agents with another ingredient selected from the group consisting of perfumes, nutraceuticals, cosmetics, biocide actives.

According to a particular embodiment, the hydrophobic material comprises a perfume.

According to a particular embodiment, the hydrophobic material consists of a perfume.

According to a particular embodiment, the hydrophobic material consists of biocide actives.

According to a particular embodiment, the hydrophobic material consists of pest control agents.

By “perfume” (or also “perfume oil”) what is meant here is an ingredient or a composition that is a liquid at about 20° C. According to any one of the above embodiments said perfume oil can be a perfuming ingredient alone or a mixture of ingredients in the form of a perfuming composition. As a “perfuming ingredient” it is meant here a compound, which is used for the primary purpose of conferring or modulating an odor. In other words such an ingredient, to be considered as being a perfuming one, must be recognized by a person skilled in the art as being able to at least impart or modify in a positive or pleasant way the odor of a composition, and not just as having an odor. For the purpose of the present invention, perfume oil also includes a combination of perfuming ingredients with substances which together improve, enhance or modify the delivery of the perfuming ingredients, such as perfume precursors, modulators, emulsions or dispersions, as well as combinations which impart an additional benefit beyond that of modifying or imparting an odor, such as long-lastingness, blooming, malodor counteraction, antimicrobial effect, microbial stability, pest control.

The nature and type of the perfuming ingredients present in the oil phase do not warrant a more detailed description here, which in any case would not be exhaustive, the skilled person being able to select them on the basis of its general knowledge and according to intended use or application and the desired organoleptic effect. In general terms, these perfuming ingredients belong to chemical classes as varied as alcohols, aldehydes, ketones, esters, ethers, acetates, nitriles, terpenoids, nitrogenous or sulfurous heterocyclic compounds and essential oils (for example Thyme oil), and said perfuming co-ingredients can be of natural or synthetic origin. Many of these co-ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery.

In particular one may cite perfuming ingredients which are commonly used in perfume formulations, such as:
Aldehydic ingredients: decanal, dodecanal, 2-methyl-undecanal, 10-undecenal, octanal, nonanal and/or nonenal;
Aromatic-herbal ingredients: eucalyptus oil, camphor, eucalyptol, 5-methyltricyclo[6.2.1.0^{2,7}]undecan-4-one, 1-methoxy-3-hexanethiol, 2-ethyl-4,4-dimethyl-1,3-oxathiane, 2,2,7/8,9/10-tetramethylspiro[5.5]undec-8-en-1-one, menthol and/or alpha-pinene;
Balsamic ingredients: coumarin, ethylvanillin and/or vanillin;
Citrus ingredients: dihydromyrcenol, citral, orange oil, linalyl acetate, citronellyl nitrile, orange terpenes, limonene, 1-p-menthen-8-yl acetate and/or 1,4 (8)-p-menthadiene;
Floral ingredients: methyl dihydrojasmonate, linalool, citronellol, phenylethanol, 3-(4-tert-butylphenyl)-2-methylpropanal, hexylcinnamic aldehyde, benzyl acetate, benzyl salicylate, tetrahydro-2-isobutyl-4-methyl-4 (2H)-pyranol, beta ionone, methyl 2-(methylamino)benzoate, (E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, 1-(2,6,6-trimethyl-1,3-cyclohexadien-1-yl)-2-buten-1-one, (2E)-1-(2,6,6-trimethyl-2-cyclohexen-1-yl)-2-buten-1-one, (2E)-1-[2,6,6-trimethyl-3-cyclohexen-1-yl]-2-buten-1-one, (2E)-1-(2,6,6-trimethyl-1-cyclohexen-1-yl)-2-buten-1-one, 3-(3,3/1,1-dimethyl-5-indanyl)propanal, 2,5-dimethyl-2-indanmethanol, 2,6,6-trimethyl-3-cyclohexene-1-carboxylate, 3-(4,4-dimethyl-1-cyclohexen-1-yl)propanal, hexyl salicylate, 3,7-dimethyl-1,6-nonadien-3-ol, 3-(4-isopropylphenyl)-2-methylpropanal, verdyl acetate, geraniol, p-menth-1-en-8-ol, 4-(1,1-dimethylethyl)-1-cyclohexyle acetate, 1,1-dimethyl-2-phenylethyl acetate, 4-cyclohexyl-2-methyl-2-butanol, amyl salicylate, high cis methyl dihydrojasmonate, 3-methyl-5-phenyl-1-pentanol, verdyl proprionate, geranyl acetate, tetrahydro linalool, cis-7-p-menthanol, propyl(S)-2-(1,1-dimethylpropoxy)propanoate, 2-methoxynaphthalene, 2,2,2-trichloro-1-phenylethyl acetate, 4/3-(4-hydroxy-4-methylpentyl)-3-cyclohexene-1-carbaldehyde, amylcinnamic aldehyde, 8-decen-5-olide, 4-phenyl-2-butanone, isononyle acetate, 4-(1,1-dimethylethyl)-1-cyclohexyl acetate, verdyl isobutyrate and/or mixture of methylionones isomers;
Fruity ingredients: gamma-undecalactone, 2,2,5-trimethyl-5-pentylcyclopentanone, 2-methyl-4-propyl-1,3-oxathiane, 4-decanolide, ethyl 2-methyl-pentanoate, hexyl acetate, ethyl 2-methylbutanoate, gamma-nonalactone, allyl heptanoate, 2-phenoxyethyl isobutyrate, ethyl 2-methyl-1,3-dioxolane-2-acetate, diethyl 1,4-cyclohexanedicarboxylate, 3-methyl-2-hexen-1-yl acetate, 1-[3,3-dimethylcyclohexy]ethyl[3-ethyl-2-oxiranyl]acetate and/or diethyl 1,4-cyclohexane dicarboxylate;
Green ingredients: 2-methyl-3-hexanone (E)-oxime, 2,4-dimethyl-3-cyclohexene-1-carbaldehyde, 2-tert-butyl-1-cyclohexyl acetate, styrallyl acetate, allyl(2-methylbutoxy)acetate, 4-methyl-3-decen-5-ol, diphenyl ether, (Z)-3-hexen-1-ol and/or 1-(5,5-dimethyl-1-cyclohexen-1-yl)-4-penten-1-one;
Musk ingredients: 1,4-dioxa-5,17-cycloheptadecanedione, (Z)-4-cyclopentadecen-1-one, 3-methylcyclopentadecanone, 1-oxa-12-cyclohexadecen-2-one, 1-oxa-13-cyclohexadecen-2-one, (9Z)-9-cycloheptadecen-1-one, 2-{(1S)-1-[(1R)-3,3-dimethylcyclohexyl]ethoxy}-2-oxoethyl propionate, 3-methyl-5-cyclopentadecen-1-one, 4,6,6,7,8,8-hexamethyl-1,3,4,6,7,8-hexahydrocyclopenta[g]isochromene, (1S,1′R)-2-[1-(3′,3′-dimethyl-1′-cyclohexyl) ethoxy]-2-methylpropyl propanoate, oxacyclohexadecan-2-one and/or (1S,1′R)-[1-(3′,3′-dimethyl-1′-cyclohexyl) ethoxycarbonyl]methyl propanoate;
Woody ingredients: 1-[(1RS,6SR)-2,2,6-trimethylcyclohexyl]-3-hexanol, 3,3-dimethyl-5-[(1R)-2,2,3-trimethyl-3-cyclopenten-1-yl]-4-penten-2-ol, 3,4′-dimethylspiro[oxirane-2,9′-tricyclo[6.2.1.0^{2,7}]undec[4]ene, (1-ethoxyethoxy)cyclododecane, 2,2,9,11-tetramethylspiro[5.5]undec-8-en-1-yl acetate, 1-(octahydro-2,3,8,8-tetramethyl-2-naphtalenyl)-1-ethanone, patchouli oil, terpenes fractions of patchouli oil, Clearwood®, (1′R,E)-2-ethyl-4-(2′,2′,3′-trimethyl-3′-cyclopenten-1′-yl)-2-buten-1-ol, 2-ethyl-4-(2,2,3-trimethyl-3-cyclopenten-1-yl)-2-buten-1-ol, methyl cedryl ketone, 5-(2,2,3-trimethyl-3-cyclopentenyl)-3-methylpentan-2-ol, 1-(2,3,8,8-tetramethyl-1,2,3,4,6,7,8,8a-octahydronaphthalen-2-yl) ethan-1-one and/or isobornyl acetate;
Other ingredients (e.g. amber, powdery spicy or watery): dodecahydro-3a,6,6,9a-tetramethyl-naphtho[2,1-b]furan and any of its stereoisomers, heliotropin, anisic aldehyde, eugenol, cinnamic aldehyde, clove oil, 3-(1,3-benzodioxol-5-yl)-2-methylpropanal, 7-methyl-2H-1,5-benzodioxepin-3 (4H)-one, 2,5,5-trimethyl-1,2,3,4,4a,5,6,7-octahydro-2-naphthalenol, 1-phenylvinyl acetate, 6-methyl-7-oxa-1-thia-4-azaspiro[4.4]nonane and/or 3-(3-isopropyl-1-phenyl) butanal.
   It is also understood that said ingredients may also be compounds known to release in a controlled manner various types of perfuming compounds also known as properfume or profragrance. Non-limiting examples of suitable properfumes may include 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, 3-(dodecylthio)-1-(2,6,6-trimethyl-3-cyclohexen-1-yl)-1-butanone, 2-(dodecylthio) octan-4-one, 2-phenylethyl oxo (phenyl)acetate, 3,7-dimethylocta-2,6-dien-1-yl oxo (phenyl)acetate, (Z)-hex-3-en-1-yl oxo (phenyl)acetate, 3,7-dimethyl-2,6-octadien-1-yl hexadecanoate, bis(3,7-dimethylocta-2,6-dien-1-yl) succinate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy) ethoxy)benzene, 2-methyl-1-(octan-3-yloxy)undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl)naphthalene, (2-phenethoxyvinyl)benzene, 2-(1-((3,7-dimethyloct-6-en-1-yl)oxy)prop-1-en-2-yl)naphthalene, (2-((2-pentylcyclopentylidene)methoxy)ethyl)benzene, 4-allyl-2-methoxy-1-((2-methoxy-2-phenylvinyl)oxy)benzene, (2-((2-heptylcyclopentylidene)methoxy)ethyl)benzene, 1-isopropyl-4-methyl-2-((2-pentylcyclopentylidene)methoxy)benzene, 2-methoxy-1-((2-pentylcyclopentylidene)methoxy)-4-propylbenzene, 3-methoxy-4-((2-methoxy-2-phenylvinyl)oxy)benzaldehyde, 4-((2-(hexyloxy)-2-phenylvinyl)oxy)-3-methoxybenzaldehyde or a mixture thereof.

The perfuming ingredients may be dissolved in a solvent of current use in the perfume industry. The solvent is preferably not an alcohol. Examples of such solvents are diethyl phthalate, isopropyl myristate, Abalyn® (rosin resins, available from Eastman), benzyl benzoate, ethyl citrate, triethyl citrate, limonene or other terpenes, or isoparaffins. Preferably, the solvent is very hydrophobic and highly sterically hindered, like for example Abalyn® or benzyl benzoate. Preferably the perfume comprises less than 30% of solvent. More preferably the perfume comprises less than 20% and even more preferably less than 10% of solvent, all these percentages being defined by weight relative to the total weight of the perfume. Most preferably, the perfume is essentially free of solvent.

Preferred perfuming ingredients are those having a high steric hindrance (bulky materials) and in particular those from one of the following groups:
Group 1: perfuming ingredients comprising a cyclohexane, cyclohexene, cyclohexanone or cyclohexenone ring substituted with at least one linear or branched C₁ to C₄ alkyl or alkenyl substituent;
Group 2: perfuming ingredients comprising a cyclopentane, cyclopentene, cyclopentanone or cyclopentenone ring substituted with at least one linear or branched C₄ to C₈ alkyl or alkenyl substituent;
Group 3: perfuming ingredients comprising a phenyl ring or perfuming ingredients comprising a cyclohexane, cyclohexene, cyclohexanone or cyclohexenone ring substituted with at least one linear or branched C₅ to C₈ alkyl or alkenyl substituent or with at least one phenyl substituent and optionally one or more linear or branched C₁ to C₃ alkyl or alkenyl substituents;
Group 4: perfuming ingredients comprising at least two fused or linked C₅ and/or C₆ rings;
Group 5: perfuming ingredients comprising a camphor-like ring structure;
Group 6: perfuming ingredients comprising at least one C₇ to C₂₀ ring structure;
Group 7: perfuming ingredients having a log P value above 3.5 and comprising at least one tert-butyl or at least one trichloromethyl substitutent;

Examples of ingredients from each of these groups are:
Group 1:2,4-dimethyl-3-cyclohexene-1-carbaldehyde (origin: Firmenich SA, Geneva, Switzerland), isocyclocitral, menthone, isomenthone, methyl 2,2-dimethyl-6-methylene-1-cyclohexanecarboxylate (origin: Firmenich SA, Geneva, Switzerland), nerone, terpineol, dihydroterpineol, terpenyl acetate, dihydroterpenyl acetate, dipentene, eucalyptol, hexylate, rose oxide, (S)-1,8-p-menthadiene-7-ol (origin: Firmenich SA, Geneva, Switzerland), 1-p-menthene-4-ol, (1RS,3RS,4SR)-3-p-mentanyl acetate, (1R,2S,4R)-4,6,6-trimethyl-bicyclo[3,1,1]heptan-2-ol, tetrahydro-4-methyl-2-phenyl-2H-pyran (origin: Firmenich SA, Geneva, Switzerland), cyclohexyl acetate, cyclanol acetate, 1,4-cyclohexane diethyldicarboxylate (origin: Firmenich SA, Geneva, Switzerland), (3RS,3aRS,6SR,7ASR)-perhydro-3,6-dimethyl-benzo[B]furan-2-one (origin: Firmenich SA, Geneva, Switzerland), ((6R)-perhydro-3,6-dimethyl-benzo[B]furan-2-one (origin: Firmenich SA, Geneva, Switzerland), 2,4,6-trimethyl-4-phenyl-1,3-dioxane, 2,4,6-trimethyl-3-cyclohexene-1-carbaldehyde;
Group 2: (E)-3-methyl-5-(2,2,3-trimethyl-3-cyclopenten-1-yl)-4-penten-2-ol (origin: Givaudan SA, Vernier, Switzerland), (1′R,E)-2-ethyl-4-(2′,2′,3′-trimethyl-3′-cyclopenten-1′-yl)-2-buten-1-ol (origin: Firmenich SA, Geneva, Switzerland), (1′R,E)-3,3-dimethyl-5-(2′,2′,3′-trimethyl-3′-cyclopenten-1′-yl)-4-penten-2-ol (origin: Firmenich SA, Geneva, Switzerland), 2-heptylcyclopentanone, methyl-cis-3-oxo-2-pentyl-1-cyclopentane acetate (origin: Firmenich SA, Geneva, Switzerland), 2,2,5-trimethyl-5-pentyl-1-cyclopentanone (origin: Firmenich SA, Geneva, Switzerland), 3,3-dimethyl-5-(2,2,3-trimethyl-3-cyclopenten-1-yl)-4-penten-2-ol (origin: Firmenich SA, Geneva, Switzerland), 3-methyl-5-(2,2,3-trimethyl-3-cyclopenten-1-yl)-2-pentanol (origin, Givaudan SA, Vernier, Switzerland);
Group 3: damascones, 1-(5,5-dimethyl-1-cyclohexen-1-yl)-4-penten-1-one (origin: Firmenich SA, Geneva, Switzerland), (1′R)-2-[2-(4′-methyl-3′-cyclohexen-1′-yl)propyl]cyclopentanone, alpha-ionone, beta-ionone, damascenone, mixture of 1-(5,5-dimethyl-1-cyclohexen-1-yl)-4-penten-1-one and 1-(3,3-dimethyl-1-cyclohexen-1-yl)-4-penten-1-one (origin: Firmenich SA, Geneva, Switzerland), 1-(2,6,6-trimethyl-1-cyclohexen-1-yl)-2-buten-1-one (origin: Firmenich SA, Geneva, Switzerland), (1S,1′R)-[1-(3′,3′-Dimethyl-1′-cyclohexyl) ethoxycarbonyl]methyl propanoate (origin: Firmenich SA, Geneva, Switzerland), 2-tert-butyl-1-cyclohexyl acetate (origin: International Flavors and Fragrances, USA), 1-(2,2,3,6-tetramethyl-cyclohexyl)-3-hexanol (origin: Firmenich SA, Geneva, Switzerland), trans-1-(2,2,6-trimethyl-1-cyclohexyl)-3-hexanol (origin: Firmenich SA, Geneva, Switzerland), (E)-3-methyl-4-(2,6,6-trimethyl-2-cyclohexen-1-yl)-3-buten-2-one, terpenyl isobutyrate, 4-(1,1-dimethylethyl)-1-cyclohexyl acetate (origin: Firmenich SA, Geneva, Switzerland), 8-methoxy-1-p-menthene, (1S,1′R)-2-[1-(3′,3′-dimethyl-1′-cyclohexyl) ethoxy]-2-methylpropyl propanoate (origin: Firmenich SA, Geneva, Switzerland), para tert-butylcyclohexanone, menthenethiol, 1-methyl-4-(4-methyl-3-pentenyl)-3-cyclohexene-1-carbaldehyde, allyl cyclohexylpropionate, cyclohexyl salicylate, 2-methoxy-4-methylphenyl methyl carbonate, ethyl 2-methoxy-4-methylphenyl carbonate, 4-ethyl-2-methoxyphenyl methyl carbonate;
Group 4: Methyl cedryl ketone (origin: International Flavors and Fragrances, USA), a mixture of (1RS,2SR,6RS,7RS,8SR)-tricyclo[5.2.1.0^{2,6}]dec-3-en-8-yl 2-methylpropanoate and (1RS,2SR,6RS,7RS,8SR)-tricyclo[5.2.1.0^{2,6}]dec-4-en-8-yl 2-methylpropanoate, vetyverol, vetyverone, 1-(octahydro-2,3,8,8-tetramethyl-2-naphtalenyl)-1-ethanone (origin: International Flavors and Fragrances, USA), (5RS,9RS,10SR)-2,6,9,10-tetramethyl-1-oxaspiro[4.5]deca-3,6-diene and the (5RS,9SR,10RS) isomer, 6-ethyl-2,10,10-trimethyl-1-oxaspiro[4.5]deca-3,6-diene, 1,2,3,5,6,7-hexahydro-1,1,2,3,3-pentamethyl-4-indenone (origin: International Flavors and Fragrances, USA), a mixture of 3-(3,3-dimethyl-5-indanyl)propanal and 3-(1,1-dimethyl-5-indanyl)propanal (origin: Firmenich SA, Geneva, Switzerland), 3′,4-dimethyl-tricyclo[6.2.1.0(2,7)]undec-4-ene-9-spiro-2′-oxirane (origin: Firmenich SA, Geneva, Switzerland), 9/10-ethyldiene-3-oxatricyclo[6.2.1.0(2,7)]undecane, (perhydro-5,5,8A-trimethyl-2-naphthalenyl acetate (origin: Firmenich SA, Geneva, Switzerland), octalynol, (dodecahydro-3a,6,6,9a-tetramethyl-naphtho[2,1-b]furan, origin: Firmenich SA, Geneva, Switzerland), tricyclo[5.2.1.0(2,6)]dec-3-en-8-yl acetate and tricyclo[5.2.1.0(2,6)]dec-4-en-8-yl acetate as well as tricyclo[5.2.1.0(2,6)]dec-3-en-8-yl propanoate and tricyclo[5.2.1.0(2,6)]dec-4-en-8-yl propanoate, (+)-(1S,2S,3S)-2,6,6-trimethyl-bicyclo[3.1.1]heptane-3-spiro-2′-cyclohexen-4′-one;
Group 5: camphor, borneol, isobornyl acetate, 8-isopropyl-6-methyl-bicyclo[2.2.2]oct-5-ene-2-carbaldehyde, pinene, camphene, 8-methoxycedrane, (8-methoxy-2,6,6,8-tetramethyl-tricyclo[5.3.1.0(1,5)]undecane (origin: Firmenich SA, Geneva, Switzerland), cedrene, cedrenol, cedrol, mixture of 9-ethylidene-3-oxatricyclo[6.2.1.0(2,7)]undecan-4-one and 10-ethylidene-3-oxatricyclo[6.2.1.0^{2,7}]undecan-4-one (origin: Firmenich SA, Geneva, Switzerland), 3-methoxy-7,7-dimethyl-10-methylene-bicyclo[4.3.1]decane (origin: Firmenich SA, Geneva, Switzerland);
Group 6: (trimethyl-13-oxabicyclo-[10.1.0]-trideca-4,8-diene (origin: Firmenich SA, Geneva, Switzerland), 9-hexadecen-16-olide (origin: Firmenich SA, Geneva, Switzerland), pentadecenolide (origin: Firmenich SA, Geneva, Switzerland), 3-methyl-(4/5)-cyclopentadecenone, (origin: Firmenich SA, Geneva, Switzerland), 3-methylcyclopentadecanone (origin: Firmenich SA, Geneva, Switzerland), pentadecanolide (origin: Firmenich SA, Geneva, Switzerland), cyclopentadecanone (origin: Firmenich SA, Geneva, Switzerland), 1-ethoxyethoxy)cyclododecane (origin: Firmenich SA, Geneva, Switzerland), 1,4-dioxacycloheptadecane-5,17-dione, 4,8-cyclododecadien-1-one;
Group 7: (+−)-2-methyl-3-[4-(2-methyl-2-propanyl)phenyl]propanal (origin: Givaudan SA, Vernier, Switzerland), 2,2,2-trichloro-1-phenylethyl acetate.

Preferably, the perfume comprises at least 30%, preferably at least 50%, more preferably at least 60% of ingredients selected from Groups 1 to 7, as defined above. More preferably said perfume comprises at least 30%, preferably at least 50% of ingredients from Groups 3 to 7, as defined above. Most preferably said perfume comprises at least 30%, preferably at least 50% of ingredients from Groups 3, 4, 6 or 7, as defined above.

According to another preferred embodiment, the perfume comprises at least 30%, preferably at least 50%, more preferably at least 60% of ingredients having a log P above 3, preferably above 3.5 and even more preferably above 3.75.

According to a particular embodiment, the perfume used in the invention contains less than 10% of its own weight of primary alcohols, less than 15% of its own weight of secondary alcohols and less than 20% of its own weight of tertiary alcohols. Advantageously, the perfume used in the invention does not contain any primary alcohols and contains less than 15% of secondary and tertiary alcohols.

According to an embodiment, the oil phase (or the oil-based core) comprises:
25-100 wt %, preferably 25-98% of a perfume oil comprising at least 15 wt % of high impact perfume raw materials having a Log T<−4, and
0-75 wt %, preferably 2-75% of a density balancing material having a density greater than 1.07 g/cm³.
   “High impact perfume raw materials” should be understood as perfume raw materials having a Log T<−4. The odor threshold concentration of a chemical compound is determined in part by its shape, polarity, partial charges and molecular mass. For convenience, the odor threshold concentration is presented as the common logarithm of the threshold concentration, i.e., Log [Threshold] (“Log T”).
   A “density balancing material” should be understood as a material having a density greater than 1.07 g/cm³ and having preferably low or no odor.
   The odor threshold concentration of a perfuming compound is determined by using a gas chromatograph (“GC”). Specifically, the gas chromatograph is calibrated to determine the exact volume of the perfume oil ingredient injected by the syringe, the precise split ratio, and the hydrocarbon response using a hydrocarbon standard of known concentration and chain-length distribution. The air flow rate is accurately measured and, assuming the duration of a human inhalation to last 12 seconds, the sampled volume is calculated. Since the precise concentration at the detector at any point in time is known, the mass per volume inhaled is known and hence the concentration of the perfuming compound. To determine the threshold concentration, solutions are delivered to the sniff port at the back-calculated concentration. A panelist sniffs the GC effluent and identifies the retention time when odor is noticed. The average across all panelists determines the odor threshold concentration of the perfuming compound. The determination of odor threshold is described in more detail in C. Vuilleumier et al., Multidimensional Visualization of Physical and Perceptual Data Leading to a Creative Approach in Fragrance Development, Perfume & Flavorist, Vol. 33, September 2008, pages 54-61.
   The nature of high impact perfume raw materials having a Log T<−4 and density balancing material having a density greater than 1.07 g/cm³ are described in WO2018115250, the content of which are included by reference.
   According to an embodiment, the high impact perfume raw materials having a Log T<−4 are selected from the group consisting of (+−)-1-methoxy-3-hexanethiol, 4-(4-hydroxy-1-phenyl)-2-butanone, 2-methoxy-4-(1-propenyl)-1-phenyl acetate, pyrazobutyle, 3-propylphenol, 1-(3-methyl-1-benzofuran-2-yl) ethanone, 2-(3-phenylpropyl)pyridine, 1-(3,3/5,5-dimethyl-1-cyclohexen-1-yl)-4-penten-1-one, 1-(5,5-dimethyl-1-cyclohexen-1-yl)-4-penten-1-one, a mixture comprising (3RS,3aRS,6SR,7ASR)-perhydro-3,6-dimethyl-benzo[b]furan-2-one and (3SR,3aRS,6SR,7ASR)-perhydro-3,6-dimethyl-benzo[b]furan-2-one, (+−)-1-(5-ethyl-5-methyl-1-cyclohexen-1-yl)-4-penten-1-one, (1'S,3′R)-1-methyl-2-[(11,2′,2′-trimethylbicyclo[3.1.0]hex-3′-yl)methyl]cyclopropyl}methanol, (+−)-3-mercaptohexyl acetate, (2E)-1-(2,6,6-trimethyl-1,3-cyclohexadien-1-yl)-2-buten-1-one, H-methyl-2 h-1,5-benzodioxepin-3 (4H)-one, (2E,6Z)-2,6-nonadien-1-ol, (4Z)-4-dodecenal, (+−)-4-hydroxy-2,5-dimethyl-3 (2H)-furanone, methyl 2,4-dihydroxy-3,6-dimethylbenzoate, 3-methylindole, (+−)-perhydro-4alpha,8abeta-dimethyl-4a-naphthalenol, patchoulol, 2-methoxy-4-(1-propenyl) phenol, mixture comprising (+−)-5,6-dihydro-4-methyl-2-phenyl-2H-pyran and tetrahydro-4-methylene-2-phenyl-2H-pyran, mixture comprising 4-methylene-2-phenyltetrahydro-2H-pyran and (+−)-4-methyl-2-phenyl-3,6-dihydro-2H-pyran, 4-hydroxy-3-methoxybenzaldehyde, nonylenic aldehyde, 2-methoxy-4-propylphenol, 3-methyl-5-phenyl-2-pentenenitrile, 1-(spiro[4.5]dec-6/7-en-7-yl)-4-penten-1-one(2-methoxynaphthalene, (−)-(3aR,5AS,9AS,9BR)-3a,6,6,9a-tetramethyldodecahydronaphtho[2,1-b]furan, 5-nonanolide, (3aR,5AS,9AS,9BR)-3a,6,6,9a-tetramethyldodecahydronaphtho[2,1-b]furan, 7-isopropyl-2H,4H-1,5-benzodioxepin-3-one, coumarin, 4-methylphenyl isobutyrate, (2E)-1-(2,6,6-trimethyl-1,3-cyclohexadien-1-yl)-2-buten-1-one, beta,2,2,3-tetramethyl-delta-methylene-3-cyclopentene-1-butanol, delta damascone ((2E)-1-[(1RS,2SR)-2,6,6-trimethyl-3-cyclohexen-1-yl]-2-buten-1-one), (+−)-3,6-dihydro-4,6-dimethyl-2-phenyl-2 h-pyran, anisaldehyde, paracresol, 3-ethoxy-4-hydroxybenzaldehyde, methyl 2-aminobenzoate, ethyl methylphenylglycidate, octalactone gamma, ethyl 3-phenyl-2-propenoate, (−)-(2E)-2-ethyl-4-[(1R)-2,2,3-trimethyl-3-cyclopenten-1-yl]-2-buten-1-ol, paracresyl acetate, dodecalactone, tricyclone, (+)-(3R,5Z)-3-methyl-5-cyclopentadecen-1-one, undecalactone, (1R,4R)-8-mercapto-3-p-menthanone, (3S,3AS,6R,7AR)-3,6-dimethylhexahydro-1-benzofuran-2 (3H)-one, beta ionone, (+−)-6-pentyltetrahydro-2H-pyran-2-one, (3E,5Z)-1,3,5-undecatriene, 10-undecenal, (9E)-9-undecenal (9Z)-9-undecenal, (Z)-4-decenal, (+−)-ethyl 2-methylpentanoate, 1,2-diallyldisulfane, 2-tridecenenitrile, 3-tridecenenitrile, (+−)-2-ethyl-4,4-dimethyl-1,3-oxathiane, (+)-(3R,5Z)-3-methyl-5-cyclopentadecen-1-one, 3-(4-tert-butylphenyl)propanal, allyl (cyclohexyloxy)acetate, methylnaphthylketone, (+−)-(4E)-3-methyl-4-cyclopentadecen-1-one, (+−)-5E3-methyl-5-cyclopentadecen-1-one, cyclopropylmethyl 3-hexenoate, (4E)-4-methyl-5-(4-methylphenyl)-4-pentenal, (+−)-1-(5-propyl-1,3-benzodioxol-2-yl) ethanone, 4-methyl-2-pentylpyridine, (+−)-(E)-3-methyl-4-(2,6,6-trimethyl-2-cyclohexen-1-yl)-3-buten-2-one, (3aRS,5aSR,9aSR,9bRS)-3a,6,6,9a-tetramethyldodecahydronaphtho[2,1-b]furan, (2S,5R)-5-methyl-2-(2-propanyl)cyclohexanone oxime, 6-hexyltetrahydro-2H-pyran-2-one, (+−)-3-(3-isopropyl-1-phenyl) butanal, methyl 2-(3-oxo-2-pentylcyclopentyl)acetate, 1-(2,6,6-trimethyl-1-cyclohex-2-enyl) pent-1-en-3-one, indol, 7-propyl-2H,4H-1,5-benzodioxepin-3-one, ethyl praline, (4-methylphenoxy) acetaldehyde, ethyl tricyclo[5.2.1.0.^{2,6}]decane-2-carboxylate, (+)-(1'S,2S,E)-3,3-dimethyl-5-(2′,2′,3′-trimethyl-3′-cyclopenten-1′-yl)-4-penten-2-ol, (4E)-3,3-dimethyl-5-[(1R)-2,2,3-trimethyl-3-cyclopenten-1-yl]-4-penten-2-ol, isopropyl-6-methyl-bicyclo[2.2.2]oct-5-ene-2-carbaldehyde, methylnonylacetaldehyde, 4-formyl-2-methoxyphenyl 2-methylpropanoate, (E)-4-decenal, (+−)-2-ethyl-4-(2,2,3-trimethyl-3-cyclopenten-1-yl)-2-buten-1-ol, (1R,5R)-4,7,7-trimethyl-6-thiabicyclo[3.2.1]oct-3-ene, (1R,4R,5R)-4,7,7-trimethyl-6-thiabicyclo[3.2.1]octane, (−)-(3R)-3,7-dimethyl-1,6-octadien-3-ol, (E)-3-phenyl-2-propenenitrile, 4-methoxybenzyl acetate, (E)-3-methyl-5-(2,2,3-trimethyl-3-cyclopenten-1-yl)-4-penten-2-ol, allyl (2/3-methylbutoxy)acetate, (+−)-(2E)-1-(2,6,6-trimethyl-2-cyclohexen-1-yl)-2-buten-1-one, (1E)-1-(2,6,6-trimethyl-1-cyclohexen-1-yl)-1-penten-3-one, and mixtures thereof.
   According to an embodiment, perfume raw materials having a Log T<−4 are chosen in the group consisting of aldehydes, ketones, alcohols, phenols, esters lactones, ethers, epoxydes, nitriles and mixtures thereof.
   According to an embodiment, perfume raw materials having a Log T<−4 comprise at least one compound chosen in the group consisting of alcohols, phenols, esters lactones, ethers, epoxydes, nitriles and mixtures thereof, preferably in amount comprised between 20 and 70% by weight based on the total weight of the perfume raw materials having a Log T<−4.
   According to an embodiment, perfume raw materials having a Log T<−4 comprise between 20 and 70% by weight of aldehydes, ketones, and mixtures thereof based on the total weight of the perfume raw materials having a Log T<−4.
   The remaining perfume raw materials contained in the oil-based core may have therefore a Log T>−4.
   According to an embodiment, the perfume raw materials having a Log T>−4 are chosen in the group consisting of ethyl 2-methylbutyrate, (E)-3-phenyl-2-propenyl acetate, (+−)-6/8-sec-butylquinoline, (+−)-3-(1,3-benzodioxol-5-yl)-2-methylpropanal, verdyl propionate, 1-(octahydro-2,3,8,8-tetramethyl-2-naphtalenyl)-1-ethanone, methyl 2-((1RS,2RS)-3-oxo-2-pentylcyclopentyl)acetate, (+−)-(E)-4-methyl-3-decen-5-ol, 2,4-dimethyl-3-cyclohexene-1-carbaldehyde, 1,3,3-trimethyl-2-oxabicyclo[2.2.2]octane, tetrahydro-4-methyl-2-(2-methyl-1-propenyl)-2H-pyran, dodecanal, 1-oxa-12/13-cyclohexadecen-2-one, (+−)-3-(4-isopropylphenyl)-2-methylpropanal, aldehyde C11, (+−)-2,6-dimethyl-7-octen-2-ol, allyl 3-cyclohexylpropanoate, (Z)-3-hexenyl acetate, 5-methyl-2-(2-propanyl)cyclohexanone, allyl heptanoate, 2-(2-methyl-2-propanyl)cyclohexyl acetate, 1,1-dimethyl-2-phenylethyl butyrate, geranyl acetate, neryl acetate, (+−)-1-phenylethyl acetate, 1,1-dimethyl-2-phenylethyl acetate, 3-methyl-2-butenyl acetate, ethyl 3-oxobutanoate, (2Z)-ethyl 3-hydroxy-2-butenoate, 8-p-menthanol, 8-p-menthanyl acetate, 1-p-menthanyl acetate, (+−)-2-(4-methyl-3-cyclohexen-1-yl)-2-propanyl acetate, (+−)-2-methylbutyl butanoate, 2-{(1S)-1-[(1R)-3,3-dimethylcyclohexyl]ethoxy}-2-oxoethyl propionate, 3,5,6-trimethyl-3-cyclohexene-1-carbaldehyde, 2,4,6-trimethyl-3-cyclohexene-1-carbaldehyde, 2-cyclohexylethyl acetate, octanal, ethyl butanoate, (+−)-(3E)-4-(2,6,6-trimethyl-1/2-cyclohexen-1-yl)-3-buten-2-one, 1-[(1RS,6SR)-2,2,6-trimethylcyclohexyl]-3-hexanol, 1,3,3-trimethyl-2-oxabicyclo[2.2.2]octane, 1,3,3-trimethyl-2-oxabicyclo[2.2.2]octane, ethyl hexanoate, undecanal, decanal, 2-phenylethyl acetate, (1S,2S,4S)-1,7,7-trimethylbicyclo[2.2.1]heptan-2-ol, (1S,2R,4S)-1,7,7-trimethylbicyclo[2.2.1]heptan-2-ol), (+−)-3,7-dimethyl-3-octanol, 1-methyl-4-(2-propanylidene)cyclohexene, (+)-(R)-4-(2-methoxypropan-2-yl)-1-methylcyclohex-1-ene, verdyl acetate, (3R)-1-[(1R,6S)-2,2,6-trimethylcyclohexyl]-3-hexanol, (3S)-1-[(1R,6S)-2,2,6-trimethylcyclohexyl]-3-hexanol, (3R)-1-[(1S,6S)-2,2,6-trimethylcyclohexyl]-3-hexanol, (+)-(1S,1′R)-2-[1-(3′,3′-dimethyl-1′-cyclohexyl) ethoxy]-2-methylpropyl propanoate, and mixtures thereof.

According to an embodiment, the perfume formulation comprises
0 to 60 wt. % of a hydrophobic solvent (based on the total weight of the perfume formulation),
40 to 100 wt. % of a perfume oil (based on the total weight of the perfume formulation), wherein the perfume oil has at least two, preferably all of the following characteristics:
   at least 35%, preferably at least 40%, preferably at least 50%, more preferably at least 60% of perfuming ingredients having a log P above 3, preferably above 3.5,
   at least 20%, preferably at least 25%, preferably at least 30%, more preferably at least 40% of Bulky materials of groups 1 to 6, preferably 3 to 6 as defined previously and
   at least 15%, preferably at least 20%, more preferably at least 25%, even more preferably at least 30% of high impact perfume materials having a Log T<−4 as defined previously, optionally, further hydrophobic active ingredients.

According to a particular embodiment, the perfume comprises 0 to 60 wt. % of a hydrophobic solvent.

According to a particular embodiment, the hydrophobic solvent is a density balancing material preferably chosen in the group consisting of benzyl salicylate, benzyl benzoate, cyclohexyl salicylate, benzyl phenylacetate, phenylethyl phenylacetate, triacetin, ethyl citrate, methyl and ethyl salicylate, benzyl cinnamate, and mixtures thereof.

In a particular embodiment, the hydrophobic solvent has Hansen Solubility Parameters compatible with entrapped perfume oil.

The term “Hansen solubility parameter” is understood refers to a solubility parameter approach proposed by Charles Hansen used to predict polymer solubility and was developed around the basis that the total energy of vaporization of a liquid consists of several individual parts. To calculate the “weighted Hansen solubility parameter” one must combine the effects of (atomic) dispersion forces, (molecular) permanent dipole-permanent dipole forces, and (molecular) hydrogen bonding (electron exchange). The weighted Hansen solubility parameter” is calculated as (δD²+δP²+δH²)^{0.5}, wherein δD is the Hansen dispersion value (also referred to in the following as the atomic dispersion fore), δP is the Hansen polarizability value (also referred to in the following as the dipole moment), and δH is the Hansen Hydrogen-bonding (“h-bonding”) value (also referred to in the following as hydrogen bonding). For a more detailed description of the parameters and values, see Charles Hansen, The Three Dimensional Solubility Parameter and Solvent Diffusion Coefficient, Danish Technical Press (Copenhagen, 1967).

Euclidean difference in solubility parameter between a fragrance and a solvent is calculated as (4*(δDₛₒₗᵥₑₙₜ−δD_{fragrance})²+(δPₛₒₗᵥₑₙₜ−δP_{fragrance})²+(δHₛₒₗᵥₑₙₜ−δH_{fragrance})²)^{0.5}, in which δDₛₒₗᵥₑₙₜ, δPₛₒₗᵥₑₙₜ, and δHₛₒₗᵥₑₙₜ, are the Hansen dispersion value, Hansen polarizability value, and Hansen h-bonding values of the solvent, respectively; and δD_{fragrance}, δP_{fragrance}, and δH_{fragrance} are the Hansen dispersion value, Hansen polarizability value, and Hansen h-bonding values of the fragrance, respectively.
In a particular embodiment, the perfume oil and the hydrophobic solvent have at least two Hansen solubility parameters selected from a first group consisting of: an atomic dispersion force (δD) from 12 to 20, a dipole moment (δP) from 1 to 8, and a hydrogen bonding (δH) from 2.5 to 11.
In a particular embodiment, the perfume oil and the hydrophobic solvent have at least two Hansen solubility parameters selected from a second group consisting of: an atomic dispersion force (δD) from 12 to 20, preferably from 14 to 20, a dipole moment (δP) from 1 to 8, preferably from 1 to 7, and a hydrogen bonding (δH) from 2.5 to 11, preferably from 4 to 11.

In a particular embodiment, at least 90% of the perfume oil, preferably at least 95% of the perfume oil, most preferably at least of 98% of the perfume oil has at least two Hansen solubility parameters selected from a first group consisting of: an atomic dispersion force (δD) from 12 to 20, a dipole moment (δP) from 1 to 8, and a hydrogen bonding (δH) from 2.5 to 11.

In a particular embodiment, the perfume oil and the hydrophobic solvent have at least two Hansen solubility parameters selected from a second group consisting of: an atomic dispersion force (δD) from 12 to 20, preferably from 14 to 20, a dipole moment (δP) from 1 to 8, preferably from 1 to 7, and a hydrogen bonding (δH) from 2.5 to 11, preferably from 4 to 11.

According to an embodiment, the perfuming formulation comprises a fragrance modulator (that can be used in addition to the hydrophobic solvent when present or as substitution of the hydrophobic solvent when there is no hydrophobic solvent).

Preferably, the fragrance modulator is defined as a fragrance material with
i. a vapor pressure of less than 0.0008 Torr at 22° C.;
ii. a c log P of 3.5 and higher, preferably 4.0 and higher and more preferably 4.5
iii. at least two Hansen solubility parameters selected from a first group consisting of: an atomic dispersion force from 12 to 20, a dipole moment from 1 to 7, and a hydrogen bonding from 2.5 to 11,
iv. at least two Hansen solubility parameters selected from a second group consisting of: an atomic dispersion force from 14 to 20, a dipole moment from 1 to 8, and a hydrogen bonding from 4 to 11, when in solution with a compound having a vapor pressure range of 0.0008 to 0.08 Torr at 22° C.

Preferably, as examples the following ingredients can be listed as modulators but the list in not limited to the following materials: alcohol C12, oxacyclohexadec-12/13-en-2-one, 3-[(2′,2′,3′-trimethyl-3′-cyclopenten-1′-yl)methoxy]-2-butanol, cyclohexadecanone, (Z)-4-cyclopentadecen-1-one, cyclopentadecanone, (8Z)-oxacycloheptadec-8-en-2-one, 2-[5-(tetrahydro-5-methyl-5-vinyl-2-furyl)-tetrahydro-5-methyl-2-furyl]-2-propanol, muguet aldehyde, 1,5,8-trimethyl-13-oxabicyclo[10.1.0]trideca-4,8-diene, (+−)-4,6,6,7,8,8-hexamethyl-1,3,4,6,7,8-hexahydrocyclopenta[g]isochromene, (+)-(1S,2S,3S,5R)-2,6,6-trimethylspiro[bicyclo[3.1.1]heptane-3,1′-cyclohexane]-2′-en-4′-one, oxacyclohexadecan-2-one, 2-{(1S)-1-[(1R)-3,3-dimethylcyclohexyl]ethoxy}-2-oxoethyl propionate, (+)-(4R,4aS,6R)-4,4a-dimethyl-6-(1-propen-2-yl)-4,4a,5,6,7,8-hexahydro-2(3H)-naphthalenone, amylcinnamic aldehyde, hexylcinnamic aldehyde, hexyl salicylate, (1E)-1-(2,6,6-trimethyl-1-cyclohexen-1-yl)-1,6-heptadien-3-one, (9Z)-9-cycloheptadecen-1-one.

According to a particular embodiment, the hydrophobic material is free of any active ingredient (such as perfume). According to this particular embodiment, it comprises, preferably consists of hydrophobic solvents, preferably chosen in the group consisting of isopropyl myristate, tryglycerides (e.g. Neobee® MCT oil, vegetable oils), D-limonene, silicone oil, mineral oil, and mixtures thereof with optionally hydrophilic solvents preferably chosen in the group consisting of 1,4-butanediol, benzyl alcohol, triethyl citrate, triacetin, benzyl acetate, ethyl acetate, propylene glycol (1,2-propanediol), 1,3-propanediol, dipropylene glycol, glycerol, glycol ethers and mixtures thereof.

The term “biocide” refers to a chemical substance capable of killing living organisms (e.g. microorganisms) or reducing or preventing their growth and/or accumulation. Biocides are commonly used in medicine, agriculture, forestry, and in industry where they prevent the fouling of, for example, water, agricultural products including seed, and oil pipelines. A biocide can be a pesticide, including a fungicide, herbicide, insecticide, algicide, molluscicide, miticide and rodenticide; and/or an antimicrobial such as a germicide, antibiotic, antibacterial, antiviral, antifungal, antiprotozoal and/or antiparasite.

As used herein, a “pest control agent” indicates a substance that serves to repel or attract pests, to decrease, inhibit or promote their growth, development or their activity. Pests refer to any living organism, whether animal, plant or fungus, which is invasive or troublesome to plants or animals, pests include insects notably arthropods, mites, spiders, fungi, weeds, bacteria and other microorganisms.

By “flavor oil”, it is meant here a flavoring ingredient or a mixture of flavoring ingredients, solvents or adjuvants of current use for the preparation of a flavoring formulation, i.e. a particular mixture of ingredients which is intended to be added to an edible composition or chewable product to impart, improve or modify its organoleptic properties, in particular its flavor and/or taste. Flavoring ingredients are well known to a person skilled in the art and their nature does not warrant a detailed description here, which in any case would not be exhaustive, the skilled flavorist being able to select them on the basis of his general knowledge and according to the intended use or application and the organoleptic effect it is desired to achieve. Many of these flavoring ingredients are listed in reference texts such as in the book by S. Arctander, Perfume and Flavor Chemicals, 1969, Montclair, N.J., USA, or its more recent versions, or in other works of similar nature such as Fenaroli's Handbook of Flavor Ingredients, 1975, CRC Press or Synthetic Food Adjuncts, 1947, by M. B. Jacobs, van Nostrand Co., Inc. Solvents and adjuvants of current use for the preparation of a flavoring formulation are also well known in the art.

In a particular embodiment, the flavor is a mint flavor. In a more particular embodiment, the mint is selected from the group consisting of peppermint and spearmint.

In a further embodiment, the flavor is a cooling agent or mixtures thereof.

In another embodiment, the flavor is a menthol flavor.

Flavors that are derived from or based on fruits where citric acid is the predominant, naturally-occurring acid include but are not limited to, for example, citrus fruits (e.g. lemon, lime), limonene, strawberry, orange, and pineapple. In one embodiment, the flavors food is lemon, lime or orange juice extracted directly from the fruit. Further embodiments of the flavor comprise the juice or liquid extracted from oranges, lemons, grapefruits, key limes, citrons, clementines, mandarins, tangerines, and any other citrus fruit, or variation or hybrid thereof. In a particular embodiment, the flavor comprises a liquid extracted or distilled from oranges, lemons, grapefruits, key limes, citrons, clementines, mandarins, tangerines, any other citrus fruit or variation or hybrid thereof, pomegranates, kiwifruits, watermelons, apples, bananas, blueberries, melons, ginger, bell peppers, cucumbers, passion fruits, mangos, pears, tomatoes, and strawberries.

In a particular embodiment, the flavor comprises a composition that comprises limonene, in a particular embodiment, the composition is a citrus that further comprises limonene.

In another particular embodiment, the flavor comprises a flavor selected from the group comprising strawberry, orange, lime, tropical, berry mix, and pineapple.

The phrase flavor includes not only flavors that impart or modify the smell of foods but include taste imparting or modifying ingredients. The latter do not necessarily have a taste or smell themselves but are capable of modifying the taste that other ingredients provides, for instance, salt enhancing ingredients, sweetness enhancing ingredients, umami enhancing ingredients, bitterness blocking ingredients and so on.

In a further embodiment, suitable sweetening components may be included in the particles described herein. In a particular embodiment, a sweetening component is selected from the group consisting of sugar (e.g., but not limited to sucrose), a stevia component (such as but not limited to stevioside or rebaudioside A), sodium cyclamate, aspartame, sucralose, sodium saccharine, and Acesulfam K or mixtures thereof.

### Water Phase Particular Embodiments

According to a particular embodiment, the process of the invention comprises the step of adding a reactant into the water phase during the reaction. This optional reactant can participate to the shell formation of microcapsules.

The reactant can be water soluble or water suspensible. Examples of suitable reactant include alcohols, amines, phenols, thiols, and mixtures thereof.

According to a particular embodiment, when the polyfunctional monomer is a polyfunctional isocyanate and no substantial amount of amines or other water-soluble reactant susceptible to polymerize with the polyfunctional isocyanate such as alcohols, thiols, ureas, urethanes, and mixtures thereof is added at any stage of the process.

According to an embodiment, the weight ratio between the oil phase and the water phase is comprised between 1:99 and 50:50, preferably between 10:90 and 40:60.

### Coacervate Particles

The coacervate particles can be “simple” coacervate particles (i.e made by “simple” coacervation) or “complex” coacervates (i.e made by “complex” coacervation) or mixtures thereof. By simple coacervation, it is understood that one polymer (or copolymer) alone made to undergo phase separation and is then precipitated from an uniform solution in the presence of anti-solvent to form the coacervate particles. By complex coacervation is understood methods in which at least two polymers (or polyelectrolyte) of opposite charge (under specific pH value) together form the coacervate particles. The complex coacervation also encompasses the formation of coacervates from a charged polyions with a polymer of oppositive charge.

### Complex Coacervate Particles

According to a particular embodiment, the coacervate is a complex coacervate particle. In other words, according to this embodiment, the coacervate particle comprises at least a first polymer and a second polymer. According to an embodiment, the first polymer and the second polymer are water-soluble.

First polymer and second polymer can also be named respectively first polyelectrolyte and second polyelectrolyte.

According to an embodiment, the first and/or second polymer are (is) biopolymer(s).

According to an embodiment, the first polymer contains at least one cationic group such as amino group, azole group, amide group, quaternary ammonium cation or guanidinium cation.

The first polymer is preferably chosen in the group consisting of proteins (such as gelatin, whey protein, albumin), chitosan and derivatives, cationic modified polysaccharides, polyethyleneimine, poly(amidoamine)s, poly(amino-co-ester), cationic polyacrylate or mixtures thereof.

According to an embodiment, the protein is a plant-protein preferably chosen in the group consisting of potato protein, chickpea protein, pea protein, faba bean protein, barley protein, oat protein, wheat gluten protein, lupin protein, canola protein, rice protein, sunflower seed protein, and mixtures thereof.

The second polymer is selected from the group of a polymer consisting of modified starch, gum arabic, chitosan, alginate salts, cellulose derivatives, guar gum, pectinate salts, pectin, carrageenan, sodium caseinate, hyaluronic acid, polyacrylic and methacrylic acid, xanthan gum or polyanion preferably chosen in the group consisting of sodium triphosphate, trisodium trimetaphosphate, sodium pyrophosphate, dextran sulfate sodium salt and mixtures thereof.

The weight ratio between the first polymer and the second polymer is preferably comprised between 1:9 and 9:1, more preferably between 3:7 and 7:3.

According to an embodiment, the coacervate particles comprise chitosan (as first polymer) and sodium caseinate (as second polymer).

According to an embodiment, the coacervate particles comprise whey protein (as first polymer) and gum arabic (as second polymer).

According to an embodiment, the coacervate particles comprise whey protein (as first polymer) and chitosan (as second polymer).

According to an embodiment, the coacervate particles comprise chitosan (as first polymer) and sodium alginate (as second polymer).

According to an embodiment, the coacervate particles comprise chitosan (as first polymer) and pectin (as second polymer).

According to an embodiment, the coacervate particle comprises a polymer and an oppositely charged polyions with the polymer (such as anions and cations). Examples of anions are polyvalent anions, such as sodium triphosphate, trisodium trimetaphosphate, sodium pyrophosphate, dextran sulfate sodium salt. Examples of cations are multivalent metal cation, such as Sn⁴⁺, Al³⁺, Fe³⁺, Sb³⁺, Ga³⁺, Ca²⁺, Mg²⁺, Zn²⁺, Ba²⁺, Cd²⁺, Co²⁺, Cu²⁺ and mixtures thereof.

According to a particular embodiment, the coacervate particles are prepared by flash nanoprecipitation (FNP). Coacervates particles can be prepared using a MIVM (Multi-Inlet-Vortex-Mixer) device which contains typically between 2 and 4 inlets.

It has been found that the preparation of coacervate particles using Flash Nanoprecipitation method (FNP) shows several advantages. Indeed, coacervate particles with controlled size and narrow size distribution can be obtained when prepared via FNP, which are one of the key factors on surface activity of these coacervate particles at the oil-in-water interface for preparing a stable Pickering emulsion.

Furthermore, when biopolymer(s) is/are used as polyelectrolyte(s), FNP can provide coacervate particle suspension with higher biopolymer content, meaning that more biopolymer can be embedded into the shell during the shell formation process.

Lastly, it is possible to include active ingredients into the coacervate particles during FNP process, providing therefore additional benefits to microcapsules.

According to an embodiment, when the coacervate particles are complex coacervate particles (i.e comprising at least two polymers), the coacervate particles are prepared by a flash nanoprecipitation (FNP) process comprising the steps of:
(i) Injection of a first polymer solution in a first stream of a multi-inlet vortex mixer,
(ii) Injection of a second polymer solution in a second stream of a multi-inlet vortex mixer
(iii) Optionally, injection of a third solution and/or a fourth solution in a third and/or forth stream of a multi-inlet vortex mixer,
(iv) Mixing the solutions injected from the different stream to form a complex coacervate particle suspension.
   First polymer and second polymer are defined as previously.
   The third solution and/or the fourth solution typically comprises water of a buffer solution.
   The buffer solution can be added in step (iii) to adjust the pH, typically between 2.0 and 8.0
   The fourth solution and/or the third solution can comprise an active ingredient. The third and/or fourth solution is preferably water miscible.
   The person skilled in the art will be able to select suitable process parameters. The injection is typically made using the pump (for example PHD ULTRA, Harvard) under a speed comprised between 10 mL/min and 120 mL/min, preferably between 20 mL/min and 80 mL/min.
   A typical process is represented on FIG. 2.

### Simple Coacervate Particles

According to a particular embodiment, the coacervate is a simple coacervate particles. In other words, according to this embodiment, the coacervate particle comprises only:
(a) water soluble polymer (or copolymer): coacervate particles formed via the precipitation from water soluble polymer solution in the presence of an anti-solvent, or
(b) water insoluble polymer (or copolymer): coacervate particles formed via the precipitation from water insoluble polymer solution in the presence of water or aqueous solution (water solution).

Simple coacervation particles may be obtained by a flash nanoprecipitation process comprising the steps of:
(i) Injection of a polymer solution in a first stream of a multi-inlet vortex mixer,
(ii) Injection of an anti-solvent in a second stream of a multi-inlet vortex mixer
(iii) Optionally, injection of a third solution and/or a fourth solution in a third and/or forth stream of a multi-inlet vortex mixer,
(iv) Mixing the solutions injected from the different streams to form a simple coacervate particle suspension.
   Depending on the nature of the polymer used in step i) (water soluble or non-water soluble), the person skilled in the art will be able to select a suitable anti-solvent in step ii).

### (a) Water-Soluble Polymers (or Co-Polymers)

A “water soluble polymer” is intended for the purpose of the present invention as encompassing any polymer which forms a one-phase solution in water. Preferably, it forms a one phase solution when dissolved in water at concentrations as high as 20% by weight, more preferably even as high as 50% by weight. Most preferably it forms a one phase solution when dissolved in water at any concentration.

The water soluble polymer may be selected from the group consisting of proteins (such as gelatin, sodium caseinate, whey protein, albumin), chitosan and derivatives, cationic modified polysaccharides, polyethyleneimine, poly(amidoamine)s, poly(amino-co-ester), cationic polyacrylate, modified starch, gum arabic, chitosan, alginate salts, zein, cellulose derivatives, guar gum, pectinate salts, pectin, carrageenan, hyaluronic acid, polyacrylic and methacrylic acid, xanthan gum or polyanion consisting of sodium triphosphate, trisodium trimetaphosphate, sodium pyrophosphate, dextran sulfate sodium salt, poly(ethylene glycol), polyvinyl alcohol, polyvinylpyrrolidone, polyacrylamide, polyethylene oxide, polyhydroxyethylmethacrylate and other hydrophilic polyacrylates, poly(N-isopropylacrylamide), polyols, polyoxazoline, polyphosphates, polyphosphazenes, polyvinyl methyl ether, locust bean gum, xyloglucan, hydroxypropyl guar, hydroxypropyl cellulose and hydroxypropyl methyl cellulose. According to an embodiment, the water soluble co-polymer containing hydrophilic block in the co-polymer, hydrophilic block could select from polyvinyl alcohol, poly(ethylene glycol), polyethylene oxide, polyacrylates, polyacrylamide, polyols, polyvinylpyrrolidone or other polyelectrolytes.

As anti-solvent, one may cite for example water miscible organic solvent, acid solution, alkaline solution or salt solution. Optionally, it may contain a further step to remove the organic solvent from the particle suspension.

### (b) Water-Insoluble Polymers (or Co-Polymers)

The polymer (or copolymer) may include proteins, polycaprolactone polyol, poly lactic-co-glycolic acid, polylactic acid, polyepoxides, polyanhydrides, poly(meth)acrylates, and co-polymer containing block of poly lactic-co-glycolic acid, polylactic acid, polyvinyl alcohol, poly(ethylene glycol), polyethylene oxide, polyacrylates, polyacrylamide, polyols, polyvinylpyrrolidone.

The water-insoluble polymer or copolymer should be dissolved in step i) in an organic solvent which is soluble with water. The water soluble solvents include methanol, ethanol, ethylene glycol, propylene glycol, glycerin, acetone, ethyl acetate, tetrahydrofuran, dimethylacetamide, dimethylformamide, dimethyl sulfoxide, acetonitrile, pyridine, acetic acid, 2-methoxyethanol, 2-ethoxyethanol, morpholine, N-methyl-2-pyrrolidone, formamide, acetamide. The polymer or copolymer could be dissolved in these solvents and be precipitated from the solution in the presence of water or water solution as anti-solvent in step ii).

The water or water solution acts as anti-solvent. One may cite also for example acid solution, alkaline solution or salt solution.

Optionally, the process may contain a further step to remove the organic solvent from the particle suspension.

According to an embodiment, the coacervate particles have a particle size comprised between 100 nm and 10 μm, preferably between 200 nm and 5 μm.

By “particle size” it is meant an average diameter of particles based on size distribution measured by dynamic light scattering (DLS) using Zetasizer Nano ZS equipment from Malvern Instruments Ltd., UK when particles are dispersed into a water phase.

Preferably, the total amount of coacervate particles present in the water phase is comprised between 0.1 and 20 wt %, preferably between 0.2 and 10 wt %.

According to an embodiment, the coacervate particles comprise an active ingredient, preferably chosen in the group consisting of a cosmetic ingredient, skin caring ingredient, perfume ingredient, flavor ingredient, malodour counteracting ingredient, antimicrobial ingredient, pharmaceutical or agrochemical ingredient, a sanitizing ingredient, an insect repellent or attractant, and mixtures thereof.

According to an embodiment, the active ingredient is an antimicrobial ingredient. Said antimicrobial ingredient may be an antibacterial ingredient, antifungal ingredient, and/or an antiparasitic ingredient. Non-exhaustive examples of such ingredients include quaternary ammonium and polymers containing quaternary ammonium group, cathelicidin antimicrobial peptides, chitosan and derivatives, polyphenols and inorganic particles such as silver particles, titanium dioxide particles and zinc oxide particles.

When prepared by FNP, the active ingredient may be added by mixing the active ingredient (or active ingredient solution) into the polymer solution or injecting the active ingredient into one stream of a multi-inlet vortex mixer during the process.

### Optional Step: Outer Coating

According to particular embodiment, the surface of the microcapsules obtained by the process of the invention can be modified with an additional step. Monomers or polymers suitable for surface modification are selected from compounds which can form chemical bond between the monomer or polymer and the microcapsules and which can improve the compatibility between the microcapsules and a target substrate.

Thus, according to a particular embodiment of the invention, at the end of step 3) of the process, one may also add to the invention's slurry a polymer selected from the group consisting of a non-ionic polysaccharide, a cationic polymer, a polysuccinimide derivative (as described for instance in WO2021185724) and mixtures thereof to form an outer coating to the microcapsule.

Non-ionic polysaccharide polymers are well known to a person skilled in the art. Preferred non-ionic polysaccharides are selected from the group consisting of locust bean gum, xyloglucan, guar gum, hydroxypropyl guar, hydroxypropyl cellulose and hydroxypropyl methyl cellulose.

Cationic polymers are also well known to a person skilled in the art. Preferred cationic polymers have cationic charge densities of at least 0.5 meq/g, more preferably at least about 1.5 meq/g, but also preferably less than about 7 meq/g, more preferably less than about 6.2 meq/g. The cationic charge density of the cationic polymers may be determined by the Kjeldahl method as described in the US Pharmacopoeia under chemical tests for Nitrogen determination. The preferred cationic polymers are chosen from those that contain units comprising primary, secondary, tertiary and/or quaternary amine groups that can either form part of the main polymer chain or can be borne by a side substituent directly connected thereto. The weight average (Mw) molecular weight of the cationic polymer is preferably between 10,000 and 3.5M Dalton, more preferably between 50,000 and 2M Dalton.

According to a particular embodiment, one will use cationic polymers based on acrylamide, methacrylamide, N-vinylpyrrolidone, quaternized N,N-dimethylaminomethacrylate, diallyldimethylammonium chloride, quaternized vinylimidazole (3-methyl-1-vinyl-1H-imidazol-3-ium chloride), vinylpyrrolidone, acrylamidopropyltrimonium chloride, cassia hydroxypropyltrimonium chloride, guar hydroxypropyltrimonium chloride or polygalactomannan 2-hydroxypropyltrimethylammonium chloride ether, starch hydroxypropyltrimonium chloride and cellulose hydroxypropyltrimonium chloride. Preferably copolymers shall be selected from the group consisting of polyquaternium-5, polyquaternium-6, polyquaternium-7, polyquaternium10, polyquaternium-11, polyquaternium-16, polyquaternium-22, polyquaternium-28, polyquaternium-43, polyquaternium-44, polyquaternium-46, cassia hydroxypropyltrimonium chloride, guar hydroxypropyltrimonium chloride or polygalactomannan 2-hydroxypropyltrimethylammonium chloride ether, starch hydroxypropyltrimonium chloride and cellulose hydroxypropyltrimonium chloride

As specific examples of commercially available products, one may cite Salcare® SC60 (cationic copolymer of acrylamidopropyltrimonium chloride and acrylamide, origin: BASF) or Luviquat®, such as the PQ 11N, FC 550 or Style (polyquaternium-11 to 68 or quaternized copolymers of vinylpyrrolidone origin: BASF), or also the Jaguar® (C13S or C17, origin Rhodia).

According to any one of the above embodiments of the invention, there is added an amount of polymer described above comprised between about 0% and 5% w/w, or even between about 0.1% and 2% w/w, percentage being expressed on a w/w basis relative to the total weight of the slurry as obtained after step 3). It is clearly understood by a person skilled in the art that only part of said added polymers will be incorporated into/deposited on the microcapsule shell.

Another object of the invention is process for the preparation of microcapsule powder comprising the steps of:
1) suspending in water coacervate particles to form a water phase;
2) admixing at least one polyfunctional monomer with a hydrophobic material to form an oil phase;
3) adding the oil phase to the water phase and mixing them to form an oil-in-water Pickering emulsion under conditions allowing the formation of a microcapsule slurry by interfacial polymerization.
4) drying the microcapsule slurry to obtain a microcapsule powder.

Any drying method known to a skilled person in the art can be used; in particular the slurry may be spray-dried preferably in the presence of a polymeric carrier material such as polyvinyl acetate, polyvinyl alcohol, dextrines, natural or modified starch, vegetable gums, pectins, xanthanes, alginates, carragenans or cellulose derivatives to provide microcapsules in a powder form.

Microcapsule powder may also comprise free perfume.

### Microcapsules

An object of the invention is a microcapsule slurry or a microcapsule powder obtainable by the processes defined above.
Another object of the invention is a microcapsule or a microcapsules slurry comprising at least one microcapsule, said microcapsule comprising:
a core, preferably an oil-based core, comprising a hydrophobic material, preferably a perfume oil
a composite shell comprising a first material and a second material, wherein:
   the first material and the second material are different,
   the first material is coacervate particles,
   the second material is a polymeric material.

The embodiments and definitions previously, especially for the hydrophobic material, coacervate particles as described herein-above applies mutatis mutandis.

The polymeric material is preferably chosen in the group consisting of polyurea, polyester, polyurethane, polyamide, polyacrylate, polysiloxane, polycarbonate, polysulfonamide, polymers of urea and formaldehyde, melamine and formaldehyde, melamine and urea, or melamine and glyoxal and mixtures thereof.

Preferably, the weight ratio in the slurry between the first material and the second material is comprised between 1:99 and 99:1, preferably between 10:90 and 99:1.

Preferably, the second material is present in an amount less than 5%, by weight based on the total weight of the microcapsule slurry.

In a particular embodiment, the shell material is a biodegradable material.

In a particular embodiment, the shell has a biodegradability of at least 40%, preferably at least 45%, 50%, 60%, 65%, 70%, 75%, 80%, 85%, 90%, 95% or 98%, within 60 days according to OECD301F.

In a particular embodiment, the core-shell microcapsule has a biodegradability of at least 40%, preferably at least 60%, preferably at least 65%, 70%, 75%, 80%, 85%, 90%, 95% or 98% within 60 days according to OECD301F.

Thereby it is understood that the core-shell microcapsule including all components, such as the core, shell and optionally coating may have a biodegradability of at least 40%, preferably at least 60%, preferably at least 65%, 70%, 75%, 80%, 85%, 90%, 95% or 98% within 60 days according to OECD301F.

In a particular embodiment, the oil-based core, preferably perfume oil has a biodegradability of at least 40%, preferably at least 60%, preferably at least 65%, 70%, 75%, 80%, 85%, 90%, 95% or 98% within 60 days according to OECD301F.

OECD301F is a standard test method on the biodegradability from the Organization of Economic Co-operation and Development.

A typical method for extracting the shell for measuring the biodegradability is disclosed in Gasparini and all in Molecules 2020, 25,718.

According to an embodiment, the coacervate particles have a particle size comprised between 100 nm and 10 μm, preferably between 200 nm and 5 μm.

### Optional Components

When microcapsules are in the form of a slurry, the microcapsule slurry can comprise auxiliary ingredients selected from the group of thickening agents/rheology modifiers, antimicrobial agents, opacity-building agents, mica particles, salt, pH stabilizers/buffering ingredients, preferably in an amount comprised between 0 and 15% by weight based on the total weight of the slurry.

According to another embodiment, the microcapsule slurry of the invention comprises additional free (i.e non-encapsulated) perfume, preferably in an amount comprised between 5 and 50% by weight based on the total weight of the slurry.

### Multiple Microcapsules System

According to an embodiment, the microcapsules of the invention (first type of microcapsule) can be used in combination with a second type of microcapsules. Another object of the invention is a microcapsule delivery system comprising:
the microcapsules of the present invention as a first type of microcapsule, and
a second type of microcapsules, wherein the first type of microcapsule and the second type of microcapsules differ in their hydrophobic material and/or their wall material and/or their coacervate particles and/or in their coating material.
   According to a particular embodiment, the microcapsule delivery system is in the form of a slurry.

The wall of the second type of microcapsules can vary. As non-limiting examples, the polymer shell of the second type of microcapsules comprises a material selected from the group consisting of polyurea, polyurethane, polyamide, polyhydroxyalkanoates, polyacrylate, polyesters, polyaminoesters, polyepoxides, organosilicon, polycarbonate, polysulfonamide, urea formaldehyde, melamine formaldehyde resin, melamine formaldehyde resin cross-linked with polyisocyanate or aromatic polyols, melamine urea resin, melamine glyoxal resin, gelatin/gum arabic shell wall, and mixtures thereof.

The second type of microcapsule can comprise an oil-based core comprising a hydrophobic active, preferably perfume, and a composite shell comprising a first material and a second material, wherein the first material and the second material are different, the first material is a coacervate, the second material is a polymeric material. In a particular embodiment, the weight ratio between the first material and the second material is comprised between 50:50 and 99.9:0.1. In a particular embodiment, the coacervate comprises a first polyelectrolyte, preferably selected among proteins (such as gelatin), polypeptides or polysaccharides (such as chitosan), most preferably Gelatin and a second polyelectrolyte, preferably alginate salts, cellulose derivatives guar gum, pectinate salts, carrageenan, polyacrylic and methacrylic acid or xanthan gum, or yet plant gums such as acacia gum (Gum Arabic), most preferably Gum Arabic. The coacervate first material can be hardened chemically using a suitable cross-linker such as glutaraldehyde, glyoxal, formaldehyde, tannic acid or genipin or can be hardenedenzymatically using an enzyme such as transglutaminase. The second polymeric material can be selected from the group consisting of polyurea, polyurethane, polyamide, polyester, polyacrylate, organosilicon, polycarbonate, polysulfonamide, polymers of urea and formaldehyde, melamine and formaldehyde, melamine and urea, or melamine and glyoxal and mixtures thereof, preferably polyurea and/or polyurethane. The second material is preferably present in an amount less than 3 wt. %, preferably less than 1 wt. % based on the total weight of the second type of microcapsule slurry.

As non-limiting examples, the shell of the second type of microcapsules can be aminoplast-based, polyurea-based or polyurethane-based. The shell of the second type of microcapsules can also be hybrid, namely organic-inorganic such as a hybrid shell composed of at least two types of inorganic particles that are cross-linked, or yet a shell resulting from the hydrolysis and condensation reaction of a polyalkoxysilane macro-monomeric composition.

According to an aspect, the shell of the second type of microcapsules comprises an aminoplast copolymer, such as melamine-formaldehyde or urea-formaldehyde or cross-linked melamine formaldehyde or melamine glyoxal.

According to another aspect the shell of the second type of microcapsules is polyurea-based made from, for example but not limited to isocyanate-based monomers and amine-containing crosslinkers such as guanidine carbonate and/or guanazole. Certain polyurea microcapsules comprise a polyurea wall which is the reaction product of the polymerisation between at least one polyisocyanate comprising at least two isocyanate functional groups and at least one reactant selected from the group consisting of an amine (for example a water-soluble guanidine salt and guanidine); a colloidal stabilizer or emulsifier; and an encapsulated perfume. However, the use of an amine can be omitted. According to a particular aspect, the colloidal stabilizer includes an aqueous solution of between 0.1% and 0.4% of polyvinyl alcohol, between 0.6% and 1% of a cationic copolymer of vinylpyrrolidone and of a quaternized vinylimidazol (all percentages being defined by weight relative to the total weight of the colloidal stabilizer). According to another aspect, the emulsifier is an anionic or amphiphilic biopolymer, which may be, in one aspect, chosen from the group consisting of gum Arabic, soy protein, gelatin, sodium caseinate and mixtures thereof.

According to another embodiment, the microcapsule wall material of the second type of microcapsules may comprise any suitable resin and especially including melamine, glyoxal, polyurea, polyurethane, polyamide, polyester, etc. Suitable resins include the reaction product of an aldehyde and an amine, suitable aldehydes include, formaldehyde and glyoxal. Suitable amines include melamine, urea, benzoguanamine, glycoluril, and mixtures thereof. Suitable melamines include, methylol melamine, methylated methylol melamine, imino melamine and mixtures thereof. Suitable ureas include, dimethylol urea, methylated dimethylol urea, urea-resorcinol, and mixtures thereof. Suitable materials for making may be obtained from one or more of the following companies Solutia Inc. (St Louis, Missouri U.S.A.), Cytec Industries (West Paterson, New Jersey U.S.A.), Sigma-Aldrich (St. Louis, Missouri U.S.A.).

According to another embodiment, the second type of microcapsules is a one-shell aminoplast core-shell microcapsule obtainable by a process comprising the steps of:
1) admixing a perfume oil with at least a polyisocyanate having at least two isocyanate functional groups to form an oil phase;
2) dispersing or dissolving into water an aminoplast resin and optionally a stabilizer to form a dispersing phase;
3) preparing an oil-in-water dispersion, wherein the mean droplet size is comprised between 1 and 100 microns, by admixing the oil phase and the dispersing phase;
4) performing a curing step to form the wall of said microcapsule; and
5) optionally drying the final dispersion to obtain the dried core-shell microcapsule.
   According to an embodiment, the second type of microcapsules is a formaldehyde-free capsule. A typical process for the preparation of aminoplast formaldehyde-free microcapsules slurry comprises the steps of
1) preparing an oligomeric composition comprising the reaction product of, or obtainable by reacting together:
   a. a polyamine component in the form of melamine or of a mixture of melamine and at least one C₁-C₄ compound comprising two NH2 functional groups;
   b. an aldehyde component in the form of a mixture of glyoxal, a C₄₋₆ 2,2-dialkoxy-ethanal and optionally a glyoxalate, said mixture having a molar ratio glyoxal/C₄₋₆ 2,2-dialkoxy-ethanal comprised between 1/1 and 10/1; and
   c. a protic acid catalyst;
2) preparing an oil-in-water dispersion, wherein the droplet size is comprised between 1 and 600 microns, and comprising:
   a. an oil;
   b. a water medium:
   c. at least an oligomeric composition as obtained in step 1;
   d. at least a cross-linker selected amongst:
      i. C₄-C₁₂ aromatic or aliphatic di- or tri-isocyanates and their biurets, triurets, trimmers, trimethylol propane-adduct and mixtures thereof; and/or
      ii. a di- or tri-oxiran compounds of formula:

A-(oxiran-2-ylmethyl)ₙ
wherein ₙ stands for 2 or 3 and 1 represents a C₂-C₆ group optionally comprising from 2 to 6 nitrogen and/or oxygen atoms;
   e. optionally a C₁-C₄ compounds comprising two NH₂ functional groups;
3) Heating the dispersion; and
4) Cooling the dispersion.

In another particular embodiment, the second type of microcapsule comprises
an oil-based core comprising a hydrophobic active, preferably perfume,
optionally an inner shell made of a polymerized polyfunctional monomer;
a biopolymer shell comprising a protein, wherein at least one protein is cross-linked.

According to a particular embodiment, the protein is chosen in the group consisting of milk proteins, caseinate salts such as sodium caseinate or calcium caseinate, casein, whey protein, hydrolyzed proteins, gelatins, gluten, pea protein, soy protein, silk protein and mixtures thereof, preferably sodium caseinate, most preferably sodium caseinate

According to a particular embodiment, the protein comprises sodium caseinate and a globular protein, preferably chosen in the group consisting of whey protein, beta-lactoglobulin, ovalbumine, bovine serum albumin, vegetable proteins, and mixtures thereof.

The protein is preferably a mixture of sodium caseinate and whey protein.

According to a particular embodiment, the biopolymer shell comprises a crosslinked protein chosen in the group consisting of sodium caseinate and/or whey protein.

According to a particular embodiment, the second type of microcapsules slurry comprises at least one microcapsule made of:
an oil-based core comprising the hydrophobic active, preferably perfume;
an inner shell made of a polymerized polyfunctional monomer; preferably a polyisocyanate having at least two isocyanate functional groups
a biopolymer shell comprising a protein, wherein at least one protein is cross-linked; wherein the protein contains preferably a mixture comprising sodium caseinate and a globular protein, preferably whey protein,
optionally at least an outer mineral layer.

According to an embodiment, sodium caseinate and/or whey protein is (are) cross-linked protein(s).

The weight ratio between sodium caseinate and whey protein is preferably comprised between 0.01 and 100, preferably between 0.1 and 10, more preferably between 0.2 and 5.

In another particular embodiment, the second type of microcapsules is a polyamide core-shell polyamide microcapsule comprising:
an oil-based core comprising a hydrophobic active, preferably perfume, and
a polyamide shell comprising or being obtainable from:
   an acyl chloride,
   a first amino compound,
   a second amino compound,
   optionally, a carbohydrate.

According to a particular embodiment, the second type of microcapsules comprises:
an oil-based core comprising a hydrophobic active, preferably perfume, and
a polyamide shell comprising or being obtainable from:
   an acyl chloride, preferably in an amount comprised between 5 and 98%, preferably between 20 and 98%, more preferably between 30 and 85% w/w
   a first amino compound, preferably in an amount comprised between 1% and 50% w/w, preferably between 7 and 40% w/w;
   a second amino compound, preferably in an amount comprised between 1% and 50% w/w, preferably between 2 and 25% w/w
   a stabilizer, preferably a biopolymer, preferably in an amount comprised between 0 and 90%, preferably between 0.1 and 75%, more preferably between 1 and 70%,
   optionally, a carbohydrate.
      According to a particular embodiment, the second type of microcapsules comprises:
an oil-based core comprising a hydrophobic active, preferably perfume, and
a polyamide shell comprising or being obtainable from:
   an acyl chloride,
   a first amino-compound being an amino-acid, preferably chosen in the group consisting of L-Lysine, L-Arginine, L-Histidine, L-Tryptophane and/or mixtures thereof.
   a second amino-compound, preferably chosen in the group consisting of ethylene diamine, diethylene triamine, cystamine and/or mixtures thereof, and
   a biopolymer, preferably chosen in the group consisting of potato protein, chickpea protein, pea protein, algae protein, faba bean protein, barley protein, oat protein, wheat gluten protein, lupin protein, soy protein, rice protein, whey protein, white egg albumin, casein, sodium caseinate, gelatin (preferably fish gelatin), bovine serum albumin, hydrolyzed soy protein, hydrolyzed sericin, pseudocollagen, silk protein, sericin powder, gelatin and mixtures thereof,
   optionally a carbohydrate, preferably selected from the group consisting of anionic salt of alginic acid, preferably alginic acid sodium salt, pectin, lignin, anionic modified starch, carboxymethylcellulose, carrageenan and mixtures thereof.

According to another aspect, the shell of the second type of microcapsules is polyurea- or polyurethane-based. Examples of processes for the preparation of polyurea and polyurethane-based microcapsule slurry are for instance described in International Patent Application Publication No. WO2007/004166, European Patent Application Publication No. EP 2300146, and European Patent Application Publication No. EP25799. Typically a process for the preparation of polyurea or polyurethane-based microcapsule slurry include the following steps:
a) Dissolving at least one polyisocyanate having at least two isocyanate groups in an oil to form an oil phase;
b) Preparing an aqueous solution of an emulsifier or colloidal stabilizer to form a dispersing phase;
c) Adding the oil phase to the dispersing phase to form an oil-in-water dispersion, wherein the mean droplet size is comprised between 1 and 500 μm, preferably between 5 and 50 μm; and
d) Applying conditions sufficient to induce interfacial polymerisation and form microcapsules in form of a slurry.

### Perfuming Composition and Consumer Products

The microcapsules of the invention can be used in combination with active ingredients. An object of the invention is therefore a composition comprising:
(i) microcapsules or microcapsule slurry as defined above;
(ii) an active ingredient, preferably chosen in the group consisting of a cosmetic ingredient, skin caring ingredient, perfume ingredient, flavor ingredient, malodour counteracting ingredient, bactericide ingredient, fungicide ingredient, pharmaceutical or agrochemical ingredient, a sanitizing ingredient, an insect repellent or attractant, and mixtures thereof.
   The capsules of the invention show a good performance in terms of stability in challenging medium.

Another object of the present invention is a perfuming composition comprising:
(i) microcapsules or microcapsule slurry as defined above, wherein the oil comprises a perfume;
(ii) at least one ingredient selected from the group consisting of a perfumery carrier, a perfumery co-ingredient and mixtures thereof;
(iii) optionally at least one perfumery adjuvant.

As liquid perfumery carriers one may cite, as non-limiting examples, an emulsifying system, i.e. a solvent and a surfactant system, or a solvent commonly used in perfumery. A detailed description of the nature and type of solvents commonly used in perfumery cannot be exhaustive. However, one can cite as non-limiting examples solvents such as dipropyleneglycol, diethyl phthalate, isopropyl myristate, benzyl benzoate, 2-(2-ethoxyethoxy)-1-ethanol or ethyl citrate, which are the most commonly used. For the compositions which comprise both a perfumery carrier and a perfumery co-ingredient, other suitable perfumery carriers than those previously specified, can be also ethanol, water/ethanol mixtures, limonene or other terpenes, isoparaffins such as those known under the trademark Isopar® (origin: Exxon Chemical) or glycol ethers and glycol ether esters such as those known under the trademark Dowanol® (origin: Dow Chemical Company). By “perfumery co-ingredient” it is meant here a compound, which is used in a perfuming preparation or a composition to impart a hedonic effect and which is not a microcapsule as defined above. In other words such a co-ingredient, to be considered as being a perfuming one, must be recognized by a person skilled in the art as being able to at least impart or modify in a positive or pleasant way the odor of a composition, and not just as having an odor.

The nature and type of the perfuming co-ingredients present in the perfuming composition do not warrant a more detailed description here, which in any case would not be exhaustive, the skilled person being able to select them on the basis of his general knowledge and according to the intended use or application and the desired organoleptic effect. In general terms, these perfuming co-ingredients belong to chemical classes as varied as alcohols, lactones, aldehydes, ketones, esters, ethers, acetates, nitriles, terpenoids, nitrogenous or sulfurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery. It is also understood that said co-ingredients may also be compounds known to release in a controlled manner various types of perfuming compounds. Co-ingredients may be chosen in the group consisting of 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, trans-3-(dodecylthio)-1-(2,6,6-trimethyl-3-cyclohexen-1-yl)-1-butanone, 2-(dodecylthio) octan-4-one, 2-phenylethyl oxo (phenyl)acetate, 3,7-dimethylocta-2,6-dien-1-yl oxo (phenyl)acetate, (Z)-hex-3-en-1-yl oxo (phenyl)acetate, 3,7-dimethyl-2,6-octadien-1-yl hexadecanoate, bis(3,7-dimethylocta-2,6-dien-1-yl) succinate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy) ethoxy)benzene, 2-methyl-1-(octan-3-yloxy) undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl) naphthalene, (2-phenethoxyvinyl)benzene, 2-(1-((3,7-dimethyloct-6-en-1-yl)oxy)prop-1-en-2-yl) naphthalene, (2-((2-pentylcyclopentylidene)methoxy)ethyl)benzene, 4-allyl-2-methoxy-1-((2-methoxy-2-phenylvinyl)oxy)benzene, (2-((2-heptylcyclopentylidene)methoxy)ethyl)benzene, 1-isopropyl-4-methyl-2-((2-pentylcyclopentylidene)methoxy)benzene, 2-methoxy-1-((2-pentylcyclopentylidene)methoxy)-4-propylbenzene, 3-methoxy-4-((2-methoxy-2-phenylvinyl)oxy)benzaldehyde, 4-((2-(hexyloxy)-2-phenylvinyl)oxy)-3-methoxybenzaldehyde or a mixture thereof or a mixture thereof.

By “perfumery adjuvant” we mean here an ingredient capable of imparting additional added benefit such as a color, a particular light resistance, chemical stability, etc. A detailed description of the nature and type of adjuvant commonly used in perfuming bases cannot be exhaustive, but it has to be mentioned that said ingredients are well known to a person skilled in the art.

Preferably, the perfuming composition according to the invention comprises between 0.01 and 30% by weight of microcapsules as defined above.

The invention's microcapsules can advantageously be used in many application fields and used in consumer products. Microcapsules can be used in liquid form applicable to liquid consumer products as well as in powder form, applicable to powder consumer products.

According to a particular embodiment, the consumer product as defined above is liquid and comprises:
a) from 2 to 65% by weight, relative to the total weight of the consumer product, of at least one surfactant;
b) water or a water-miscible hydrophilic organic solvent; and
c) a microcapsule slurry or microcapsules as defined above,
d) optionally non-encapsulated perfume.

According to a particular embodiment, the consumer product as defined above is in a powder form and comprises:
a) from 2 to 65% by weight, relative to the total weight of the consumer product, of at least one surfactant;
b) a microcapsule powder as defined above.
c) optionally perfume powder that is different from the microcapsules defined above.

In the case of microcapsules including a perfume oil-based core, the products of the invention, can in particular be of used in perfumed consumer products such as product belonging to fine fragrance or “functional” perfumery. Functional perfumery includes in particular personal-care products including hair-care, body cleansing, skin care, hygiene-care as well as home-care products including laundry care, surface care and air care. Consequently, another object of the present invention consists of a perfumed consumer product comprising as a perfuming ingredient, the microcapsules defined above or a perfuming composition as defined above. The perfume element of said consumer product can be a combination of perfume microcapsules as defined above and free or non-encapsulated perfume, as well as other types of perfume microcapsules than those here-disclosed.

In particular a liquid consumer product comprising:
a) from 2 to 65% by weight, relative to the total weight of the consumer product, of at least one surfactant;
b) water or a water-miscible hydrophilic organic solvent; and
c) a perfuming composition as defined above is another object of the invention.

Also a powder consumer product comprising
(a) from 2 to 65% by weight, relative to the total weight of the consumer product, of at least one surfactant; and
(b) a perfuming composition as defined above is part of the invention.

The invention's microcapsules can therefore be added as such or as part of an invention's perfuming composition in a perfumed consumer product.

For the sake of clarity, it has to be mentioned that, by “perfumed consumer product” it is meant a consumer product which is expected to deliver among different benefits a perfuming effect to the surface to which it is applied (e.g. skin, hair, textile, paper, or home surface) or in the air (air-freshener, deodorizer etc.). In other words, a perfumed consumer product according to the invention is a manufactured product which comprises a functional formulation also referred to as “base”, together with benefit agents, among which an effective amount of microcapsules according to the invention.

The nature and type of the other constituents of the perfumed consumer product do not warrant a more detailed description here, which in any case would not be exhaustive, the skilled person being able to select them on the basis of his general knowledge and according to the nature and the desired effect of said product. Base formulations of consumer products in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

Non-limiting examples of suitable perfumed consumer products can be a perfume, such as a fine perfume, a cologne, an after-shave lotion, a body-splash; a fabric care product, such as a liquid or solid detergent, tablets and unit dose (single or multi-chambers), a fabric softener, a dryer sheet, a fabric refresher, an ironing water, or a bleach; a personal-care product, such as a hair-care product (e.g. a shampoo, hair conditioner, a coloring preparation or a hair spray), a cosmetic preparation (e.g. a vanishing cream, body lotion or a deodorant or antiperspirant), or a skin-care product (e.g. a perfumed soap, shower or bath mousse, body wash, oil or gel, bath salts, or a hygiene product); an air care product, such as an air freshener or a “ready to use” powdered air freshener; or a home care product, such all-purpose cleaners, liquid or power or tablet dishwashing products, toilet cleaners or products for cleaning various surfaces, for example sprays & wipes intended for the treatment/refreshment of textiles or hard surfaces (floors, tiles, stone-floors etc.); a hygiene product such as sanitary napkins, diapers, toilet paper.

Another object of the invention is a consumer product comprising:
a personal care active base, and
microcapsules or microcapsule slurry as defined above or the perfuming composition as defined above,
wherein the consumer product is in the form of a personal care composition.

Personal care active bases in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

The personal care composition is preferably chosen in the group consisting of a hair-care product (e.g. a shampoo, hair conditioner, a coloring preparation or a hair spray), a cosmetic preparation (e.g. a vanishing cream, body lotion or a deodorant or antiperspirant), or a skin-care product (e.g. a perfumed soap, shower or bath mousse, body wash, oil or gel, bath salts, or a hygiene product);

Another object of the invention is a consumer product comprising:
a home care or a fabric care active base, and
microcapsules or microcapsule slurry as defined above or the perfuming composition as defined above,
wherein the consumer product is in the form of a home care or a fabric care composition.

Home care or fabric care active bases in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

Preferably, the consumer product comprises from 0.1 to 15 wt %, more preferably between 0.2 and 5 wt % of the microcapsules of the present invention, these percentages being defined by weight relative to the total weight of the consumer product. Of course the above concentrations may be adapted according to the benefit effect desired in each product.

An object of the invention is a consumer product, preferably a home care or a fabric care consumer product comprising the microcapsules or the microcapsule slurry as defined above, wherein the consumer product has a pH less than 7.

An object of the invention is a consumer product, preferably a home care or a fabric care consumer product comprising the microcapsules or the microcapsule slurry as defined above, wherein the consumer product has a pH equals or greater than 7.

For liquid consumer product mentioned below, by “active base”, it should be understood that the active base includes active materials (typically including surfactants) and water.

For solid consumer product mention below, by “active base”, it should be understood that the active base includes active materials (typically including surfactants) and auxiliary agents (such as bleaching agents, buffering agent; builders; soil release or soil suspension polymers; granulated enzyme particles, corrosion inhibitors, antifoaming, sud suppressing agents; dyes, fillers, and mixtures thereof).

### Fabric Softener

An object of the invention is a consumer product in the form of a fabric softener composition comprising:
a fabric softener active base; preferably comprising at least one active material chosen in the group consisting of dialkyl quaternary ammonium salts, dialkyl ester quaternary ammonium salts (esterquats), Hamburg esterquat (HEQ), TEAQ (triethanolamine quat), silicones and mixtures thereof, the active base being used preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition,
optionally free perfume oil.

### Liquid Detergent

An object of the invention is a consumer product in the form of a liquid detergent composition comprising:
a liquid detergent active base; preferably comprising at least one active material chosen in the group consisting of anionic surfactant such as alkylbenzenesulfonate (ABS), secondary alkyl sulfonate (SAS), primary alcohol sulfate (PAS), lauryl ether sulfate (LES), methyl ester sulfonate (MES) and nonionic surfactant such as alkyl amines, alkanolamide, fatty alcohol poly(ethylene glycol) ether, fatty alcohol ethoxylate (FAE), ethylene oxide (EO) and propylene oxide (PO) copolymers, amine oxydes, alkyl polyglucosides, alkyl polyglucosamides, the active base being used preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition,
optionally free perfume oil.

### Solid Detergent

An object of the invention is a consumer product in the form of a solid detergent composition comprising:
a solid detergent active base; preferably comprising at least one active material chosen in the group consisting of anionic surfactant such as alkylbenzenesulfonate (ABS), secondary alkyl sulfonate (SAS), primary alcohol sulfate (PAS), lauryl ether sulfate (LES), methyl ester sulfonate (MES) and nonionic surfactant such as alkyl amines, alkanolamide, fatty alcohol poly(ethylene glycol) ether, fatty alcohol ethoxylate (FAE), ethylene oxide (EO) and propylene oxide (PO) copolymers, amine oxydes, alkyl polyglucosides, alkyl polyglucosamides, the active base being used preferably in an amount comprised between 85 and 99.95% by weight based on the total weight of the composition,
a microcapsule powder or microcapsule slurry or microcapsules as defined above, preferably in an amount comprised between 0.05 to 15 wt %, more preferably between 0.1 and 5 wt % by weight based on the total weight of the composition,
optionally free perfume oil.

### Shampoo/Shower Gel

An object of the invention is a consumer product in the form of a shampoo or a shower gel composition comprising:
a shampoo or a shower gel active base; preferably comprising at least one active material chosen in the group consisting of sodium alkylether sulfate, ammonium alkylether sulfates, alkylamphoacetate, cocamidopropyl betaine, cocamide MEA, alkylglucosides and aminoacid based surfactants and mixtures thereof, the active base being used preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition,
optionally free perfume oil.

### Rinse-Off Conditioner

An object of the invention is a consumer product in the form of a rinse-off conditioner composition comprising:
a rinse-off conditioner active base; preferably comprising at least one active material chosen in the group consisting of cetyltrimonium chloride, stearyl trimonium chloride, benzalkonium chloride, behentrimonium chloride and mixture thereof, the active base being used preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition,
optionally free perfume oil.

### Solid Scent Booster

An object of the invention is a consumer product in the form of a solid scent booster composition comprising:
a solid carrier, preferably chosen in the group consisting of urea, sodium chloride, sodium sulphate, sodium acetate, zeolite, sodium carbonate, sodium bicarbonate, clay, talc, calcium carbonate, magnesium sulfate, gypsum, calcium sulfate, magnesium oxide, zinc oxide, titanium dioxide, calcium chloride, potassium chloride, magnesium chloride, zinc chloride, saccharides such as sucrose, mono-, di-, and polysaccharides and derivatives such as starch, cellulose, methyl cellulose, ethyl cellulose, propyl cellulose, polyols/sugar alcohols such as sorbitol, maltitol, xylitol, erythritol, and isomalt, PEG, PVP, citric acid or any water soluble solid acid, fatty alcohols or fatty acids and mixtures thereof,
a microcapsule slurry or microcapsules as defined above, in a powdered form, preferably in an amount comprised between 0.05 to 15 wt %, more preferably between 0.1 and 5 wt % by weight based on the total weight of the composition,
optionally free perfume oil.

### Liquid Scent Booster

An object of the invention is a consumer product in the form of a liquid scent booster composition comprising:
an aqueous phase,
a surfactant system essentially consisting of one or more than one non-ionic surfactant, wherein the surfactant system has a mean HLB between 10 and 14, preferably chosen in the group consisting of ethoxylated aliphatic alcohols, POE/PPG (polyoxyethylene and polyoxypropylene) ethers, mono and polyglyceryl esters, sucrose ester compounds, polyoxyethylene hydroxylesters, alkyl polyglucosides, amine oxides and combinations thereof;
a linker chosen in the group consisting of alcohols, salts and esters of carboxylic acids, salts and esters of hydroxyl carboxylic acids, fatty acids, fatty acid salts, glycerol fatty acids, surfactant having an HLB less than 10 and mixtures thereof, and
a microcapsule slurry or microcapsules as defined above, in the form of a slurry, preferably in an amount comprised between 0.05 to 15 wt %, more preferably between 0.1 and 5 wt % by weight based on the total weight of the composition,
optionally free perfume oil.

### Hair Coloration

An object of the invention is a consumer product in the form of an oxidative hair coloring composition comprising:
an oxidizing phase comprising an oxidizing agent and an alkaline phase comprising an alkakine agent, a dye precursor and a coupling compound; wherein said dye precursor and said coupling compound form an oxidative hair dye in the presence of the oxidizing agent, preferably in an amount comprised between 85 and 99.95% by weight based on the total weight of the composition,
microcapsule slurry or microcapsules as defined above, preferably in an amount comprised between 0.05 to 15 wt %, more preferably between 0.1 and 5 wt % by weight based on the total weight of the composition,
optionally free perfume oil

### Perfuming Composition

According to a particular embodiment, the consumer product is in the form of a perfuming composition comprising:
0.1 to 30%, preferably 0.1 to 20% of or microcapsule slurry or microcapsules as defined previously,
0 to 40%, preferably 3-40% of perfume, and
20-90%, preferably 40-90% of ethanol, by weight based on the total weight of the perfuming composition.

The invention will now be further described by way of examples. It will be appreciated that the invention as claimed is not intended to be limited in any way by these examples.

### EXAMPLES

### Example 1

### Preparation of Microcapsules According to the Invention

TABLE 1
Perfume oil A

| Ingredients | % in oil |
|---|---|
| Ethyl 2-methyl-pentanoate | 3.20% |
| Eucalyptol | 7.80% |
| 2,4-Dimethyl-3-cyclohexene-1-carbaldehyde | 0.75% |
| Aldehyde C₁₀ | 0.75% |
| Citronellyl Nitrile | 4.30% |
| Isobornyl acetate | 3.00% |
| 2-tert-butyl-1-cyclohexyl acetate | 9.80% |
| Citronellyl Acetate | 1.30% |
| 2-Methylundecanal | 3.00% |
| Diphenyloxide | 0.80% |
| Aldehyde C₁₂ | 1.30% |
| Dicyclopentadiene acetate | 9.85% |
| Ionone beta | 3.30% |
| Undecalactone gamma | 18.75% |
| Hexyl Salicylate | 15.90% |
| Benzyl Salicylate | 16.20% |

### Microcapsules A

### Sodium Caseinate/Chitosan Complex Particle Suspension Preparation

2.0% Sodium Caseinate solution (Sodium Caseinate dissolved in DI water) and 0.4% Chitosan solution (Chitosan dissolved in 1% Acetic acid solution) were injected into two different streams of a four stream multi inlet vortex. The other two streams were injected with water with pH at 5.0. The injection was made using the pump (PHD ULTRA, Harvard) under the speed at 40 mL/min.
The obtained complex coacervation suspension contains 0.5% Sodium Caseinate and 0.1% Chitosan (mean particle size: 347 nm) with the pH comprised between 4.8-5.0.

### Microcapsules Preparation

An oil phase comprising a perfume oil (see Table 1) and Takenate® (Xylylene Diisocyanate adduct polymer) was added into the complex coacervation suspension with an oil/water ratio at 3:7. Then, a stable Pickering emulsion was formed by using a homogenizer.
The Pickering emulsion was transferred into a reactor and the interfacial reaction was carried out at 70° C. for 3 h.

TABLE 2
Microcapsules A composition

| | Effective Amount of |
|---|---|
| Ingredients | Ingredient (% wt)^{a)} |
| Water | 69.58 |
| Sodium Caseinate | 0.35 |
| Chitosan | 0.07 |
| Perfume oil | 29.4 |
| Takenate ® D-110N¹⁾ | 0.6 |

¹⁾Takenate ® D-110N (75% active solution in ethyl acetate of Trimethylol propane-adduct of xylylene diisocyanate), origin: Mitsui Chemicals, Inc., Japan

### Microcapsules B

### Sodium Caseinate/Chitosan Complex Particles Suspension Preparation

2.0% Sodium Caseinate solution (Sodium Caseinate dissolved in DI water) and 0.4% Chitosan solution (Chitosan dissolved in 1% Acetic acid solution) were injected into a two-stream inlet vortex. The injection was made using the pump (PHD ULTRA, Harvard) under the speed at 40 mL/min.
The obtained complex coacervation suspension containing 1.0% Sodium Caseinate and 0.2% Chitosan (mean particle size: 764 nm), the pH of the suspension was adjusted to 4.8-5.0.

### Microcapsules Preparation

An oil phase comprising a perfume oil (see Table 1) and Takenate® (Xylylene Diisocyanate adduct polymer was added into the complex coacervation suspension with an oil/water ratio at 3:7. Then, a stable Pickering emulsion was formed by using a homogenizer.
The Pickering emulsion was transferred into a reactor and the interfacial reaction was carried out at 70° C. for 3h.

TABLE 3
Microcapsules B composition

| | Effective Amount of |
|---|---|
| Ingredients | Ingredient (% wt)a⁾ |
| Water | 69.16 |
| Sodium Caseinate | 0.7 |
| Chitosan | 0.14 |
| Perfume oil | 29.4 |
| Xylylene Diisocyanate | 0.6 |
| adduct polymer1) | |

1) Takenate® D-110N (Trimethylol propane-adduct
of xylylene diisocyanate),
origin: Mitsui Chemicals, Inc., Japan

FIG. 2 represents microscopic picture of microcapsules B.

### Microcapsules C

### Whey Protein/Gum Arabic Complex Particles Suspension Preparation

0.6% Whey protein solution (Whey protein dissolved in DI water) and 1.2% Gum Arabic solution (Gum Arabic dissolved in DI water) were injected into a two-stream inlet vortex. The injection was made using the pump (PHD ULTRA, Harvard) under the speed at 40 mL/min. The obtained complex coacervation suspension contains 0.3% whey protein and 0.6% Gum Arabic (mean particle size: 1.2 μm) with the pH was adjusted between 4.3-4.6.
An oil phase comprising a perfume oil (see Table 1) and Takenate® (Xylylene Diisocyanate adduct polymer) was added into the complex coacervation suspension with an oil/water ratio at 3:7. Then, a stable Pickering emulsion was formed by using a homogenizer.
The Pickering emulsion was transferred into a reactor and the interfacial reaction was carried out at 70° C. for 3h.

TABLE 4
Microcapsules C composition

| | Effective Amount of |
|---|---|
| Ingredients | Ingredient (% wt)a⁾ |
| Water | 69.37 |
| Whey protein | 0.21 |
| Gum Arabic | 0.42 |
| Perfume oil | 29.4 |
| Xylylene Diisocyanate | 0.6 |
| adduct polymer1) | |

1) Takenate® D-110N (Trimethylol propane-adduct
of xylylene diisocyanate),
origin: Mitsui Chemicals, Inc., Japan

### Microcapsules D

### Whey Protein/Chitosan Complex Particles Suspension Preparation

4.0% Whey protein solution (Whey protein dissolved in DI water) and 0.8% Chitosan solution (Chitosan dissolved in 1% Acetic acid solution) were injected into two steams of a four stream multi-inlet vortex, the other two streams were injected with water with pH at 5.5. The injection was made using the pump (PHD ULTRA, Harvard) under the speed at 40 mL/min.
The obtained complex coacervation suspension containing 1.0% Whey protein and 0.2% Chitosan (mean particle size: 5.0 μm) with the pH ca. 5.5.
An oil phase comprising a perfume oil (see Table 1) and Takenate® (Xylylene Diisocyanate adduct polymer) was added into the complex coacervation suspension with an oil/water ratio at 3:7. Then, a stable Pickering emulsion was formed by using a homogenizer.
The Pickering emulsion was transferred into a reactor and the interfacial reaction was carried out at 70° C. for 3h.

TABLE 5
Microcapsules D composition

| | Effective Amount of |
|---|---|
| Ingredients | Ingredient (% wt)a⁾ |
| Water | 69.16 |
| Whey protein | 0.7 |
| Chitosan | 0.14 |
| Perfume | 29.4 |
| Xylylene Diisocyanate | 0.6 |
| adduct polymer1) | |

1) Takenate® D-110N (Trimethylol
propane-adduct of xylylene diisocyanate),
origin: Mitsui Chemicals, Inc., Japan

### Microcapsule E

### Potato Protein/Gum Arabic Complex Particle Suspension Preparation

2.0% potato protein solution (pH was adjusted to 2 by using 5.0% hydrochloric acid solution and 5.0% sodium hydroxide solution) and 2.0% gum arabic solution (pH was adjusted to 2 by using 5.0% hydrochloric acid solution and 5.0% sodium hydroxide solution) were injected into two different streams of a two stream multi-inlet vortex. The injection was made using the pump (PHD ULTRA, Harvard) under the speed at 40 mL/min.
The obtained complex coacervation suspension contains 1.0% potato protein and 1.0% gum arabic (mean particle size: 814.7 nm) with the pH comprised 2.

### Microcapsules Preparation

An oil phase comprising a perfume oil (see Table 1) and Takenate® (Xylylene Diisocyanate adduct polymer) was added into the water phase comprising complex coacervation suspension and melamine-formaldehyde resin (MF resin) with an oil/water ratio at 3:7. Then a stable Pickering emulsion was formed by using a homogenizer. The Pickering emulsion was transferred into a reactor and the interfacial reaction was carried out at 80° C. for 2 h. Then ethylene urea was added, and the slurry was cooled down to RT.

TABLE 6
Microcapsules E composition

| | Effective Amount of |
|---|---|
| Ingredients | Ingredient (% wt)a⁾ |
| Water | 63.26 |
| Potato protein1) | 0.62 |
| Gum arabic | 0.62 |
| MF resin2) | 2.5 |
| Takenate® D-110N3) | 0.6 |
| Perfume oil | 29.4 |
| Ethylene urea | 3.0 |

1) Solanic 200, origin: Royal Avebe U.A., Netherlands
2) Melamine Formaldehyde resin
3) Takenate® D-110N (75% active solution
in ethyl acetate of Trimethylol propane-
adduct of xylylene diisocyanate),
origin: Mitsui Chemicals, Inc., Japan

### Microcapsule F

### Zein Simple Particle Suspension Preparation

Zein was dissolved into ethanol/water (70/30 v/v) mixture for a solution with solid content at 2.0%. Zein solution and water were injected into two different streams of a two stream multi-inlet vortex. The injection was made using the pump (PHD ULTRA, Harvard) under the speed at 40 mL/min. The obtained suspension was evaporated by rotavapor removing ethanol to obtain a final suspension with solid content at ca. 6.8% (mean particle size: 615.1 nm).

### Microcapsules Preparation

An oil phase comprising a perfume oil (see Table 1) and ethylene glycol dimethacrylate (EGDMA) was added into the dilute zein simple coacervation suspension (2.0% zein particles in suspension) with an oil/water ratio at 3:7. Then a stable Pickering emulsion was formed by using a homogenizer, and the Pickering emulsion was transferred into a reactor. Ammonium persulfate (APS) solution and sodium bisulfite solution were added into the Pickering emulsion. Then reaction was carried out at 40° C. for 3 h and 60° C. for 1 h under N₂ atmosphere.

TABLE 7
Microcapsules F composition

| | Effective Amount of |
|---|---|
| Ingredients | Ingredient (% wt)a⁾ |
| Water | 69.67 |
| Zein | 1.34 |
| EGDMA | 2.16 |
| APS | 0.16 |
| Sodium bisulfite | 0.05 |
| Perfume oil | 26.62 |

### Example 2

### Characterization and Performance of Microcapsules According to the Invention

Size measurement: Mean size (D [4,3]) of microcapsule slurry was measured by using the Mastersizer 3000 equipment from Malvern Instruments Ltd., UK
Zeta potential measurement: Zeta potential of microcapsules was examined using Zetasizer Nano ZS from Malvern Instruments Ltd., UK

| Example | Mean size | Zeta Potential |
|---|---|---|
| A | 21.8 um | -70.0 mV |
| B | 25.8 um | -37.5 mV |
| C | 8.9 um | -37.7 mV |
| D | 39.7 um | -4.5 mV |
| E | 31.5 um | -32.7 mV |
| F | 48.9 um | -12.5 mV |

### Stability

The storage stability of the capsules was evaluated in a fabric softener formulation (see composition in Table 8). Capsule dispersions were diluted in the fabric softener (29.73 g) to obtain a final concentration of perfume of 0.20 wt %. The softener was stored for up to 3 days at 37° C. The amount of perfume leaked out of the capsules was then measured by solvent extraction with isooctane (10 mL) under stirring.

TABLE 8
Fabric softener composition

| Product | Origin | Wt % |
|---|---|---|
| Stepantex VL 90A | Stepan | 8.88 |
| Calcium Chloride Sol. 10% | | 0.36 |
| Proxel GXL | Avecia | 0.04 |
| Water | | 90.72 |
| TOTAL | | 100 |

### Protocol for the Stability Assessment

A fabric softener base comprising microcapsules (2 g) was introduced into a 20 mL vial. The sample was diluted with water (2 mL) to decrease the viscosity using a Socorex 10 ml bottle top dispenser. The samples were shaken for 5 min using the Turbulat Shaker set at 40 rpm. Isooctane, containing 1,4-dibromobenzene as an internal standard at a precisely known concentration around 90 ng/μL, was added to the vial (10 mL). The samples were shaken for 45 min at 40 rpm to extract the free perfume and then centrifuged for 10 min at 3.0 G to separate the two phases. The solvent phase was recovered and dried on MgSO₄ for GC analysis.

TABLE 9

| Perfume leakage from microcapsules in a fabric | |
|---|---|
| softener after storage at 37° C. for 3 days | |
| Microcapsule | Leakage (%) |
| A | 8.3 |
| B | 15.1 |
| C | 21.3 |
| D | 7.1 |
| E | 11.2 |

Microcapsules according to the invention show therefore a good stability in a fabric softener.

### Performance Intensity

Perfume oil dosage in the fabric softener: 0.1%

### Washing Protocol:

(1) Capsule slurry was added into 26 g unperfumed fabric softener with perfume oil dosage of 0.1% under stirring at 200 rpm for 5 min, the fabric softener was used to wash the cotton towels.
(2) Cotton towels (30 cm*30 cm—1.4 kg for 36 towels) were washed with a washing machine (with 65 g unperfumed detergent and the fabric softener containing capsules in step 1) in a regular wash cycle: water level-43 L, wash cycle and time-regular mode for 42 min, water temperature-RT @25° C.
(3) The washed towels were let dry 24 h at RT before processing to the evaluation before and after rubbing.
   A sensory panel was performed, 8 panelists were asked to rate the perfume intensity on dry towels before and after rubbing using a scale between 1 (non-perceptible perfume) and 7 (very strong perfume intensity).
   Performance intensity (scale from 1 to 7): Before rubbing 2.8±0.1; after rubbing 4.5±0.1 for microcapsules A.
   Performance intensity (scale from 1 to 7): Before rubbing 3.2±0.1; after rubbing 4.6±0.1 for microcapsules B.
   Performance intensity (scale from 1 to 7): Before rubbing 3.0±0.1; after rubbing 4.3±0.1 for microcapsules C.
   Performance intensity (scale from 1 to 7): Before rubbing 2.1±0.1; after rubbing 4.7±0.1 for microcapsules D.

### Example 3

### Liquid Detergent Composition

Microcapsules of the present invention are dispersed in a liquid detergent base described below to obtain a concentration of encapsulated perfume oil at 0.22%.

TABLE 10

| Liquid detergent composition | |
|---|---|
| | Concentration |
| Ingredients | [wt %] |
| Sodium C14-17 Alkyl | 7 |
| Sec Sulfonate¹⁾ | |
| Fatty acids, C12-18 and | 7.5 |
| C18-unsaturated²⁾ | |
| C12/14 fatty alcohol polyglycol | 17 |
| ether with 7 mol EO³⁾ | |
| Triethanolamine | 7.5 |
| Propylene Glycol | 11 |
| Citric acid | 6.5 |
| Potassium Hydroxyde | 9.5 |
| Protease | 0.2 |
| Amylase | 0.2 |
| Mannanase | 0.2 |
| Acrylates/Steareth-20 | 6 |
| Methacrylate | |
| structuring Crosspolymer4) | |
| Deionized Water | 27.4 |

1) Hostapur® SAS 60; Origin: Clariant
2) Edenor® K 12-18; Origin: Cognis
3) Genapol® LA 070; Origin: Clariant
4) Aculyn® 88; Origin: Dow Chemical

### Example 4

### Rinse-Off Conditioner

Microcapsules of the present invention are dispersed in a rinse-off conditioner base described below to obtain a concentration of encapsulated perfume oil at 0.5%.

TABLE 11
Rinse-off conditioner composition

| | | Concentration |
|---|---|---|
| | Ingredients | [wt %] |
| A | Water deionized | 81.8 |
| | Behentrimonium Chloride ¹⁾ | 2.5 |
| | Hydroxyethylcellulose ²⁾ | 1.5 |
| B | Cetearyl Alcohol ³⁾ | 4 |
| | Glyceryl Stearate (and) | 2 |
| | PEG-100 Stearate ⁴⁾ | |
| | Behentrimonium Methosulfate | 4 |
| | (and) Cetyl alcohol | |
| | (and) Butylene Glycol ⁵⁾ | |
| | Ethoxy (20) Stearyl Alcohol ⁶⁾ | 1 |
| C | Amodimethicone (and) Trideceth-12 | 3 |
| | (and) Cetrimonium Chloride ⁷⁾ | |
| | Chlorhexidine Digluconate ⁸⁾ 20% | 0.2 |
| | aqueous solution | |
| D | Citric acid 10% aqueous sol. | q.s. |
| | till pH 3.5-4 | |
| | TOTAL: | 100 |

1) Genamin KDM P, Clariant
2) Tylose H10 Y G4, Shin Etsu
3) Lanette O, BASF
4) Arlacel 165-FP-MBAL-PA-(RB), Croda
5) Incroquat Behenyl TMS-50-MBAL-PA-(MH) HA4112, Croda
6) SP Brij S20 MBAL-PA(RB), Croda
7) Xiameter DC MEM-0949 Emulsion, Dow Corning
8) Alfa Aesar

### Example 5

### Shampoo Composition

Microcapsules of the present invention are weighed and mixed in a shampoo composition to add the equivalent of 0.2% perfume.

TABLE 12
Shampoo composition

| | | Concentration |
|---|---|---|
| | Ingredients | [wt %] |
| A | Water deionized | 44.4 |
| | Polyquaternium-10 ¹⁾ | 0.3 |
| | Glycerin 85% ²⁾ | 1 |
| | DMDM Hydantoin ³⁾ | 0.2 |
| B | Sodium Laureth Sulfate ⁴⁾ | 28 |
| | Cocamidopropyl Betaine ⁵⁾ | 3.2 |
| | Disodium Cocoamphodiacetate ⁶⁾ | 4 |
| | Ethoxy (20) Stearyl Alcohol ⁶⁾ | 1 |
| C | Sodium Laureth Sulfate ⁴⁾ | 3 |
| | Glyceryl Laureate ⁷⁾ | 0.2 |
| D | Water deionized | 1 |
| | Sodium Methylparaben ⁸⁾ | 0.1 |
| E | Sodium Chloride 10% aqueous sol. | 15 |
| | Citric acid 10% aqueous sol. | q.s. |
| | till pH 5.5-6 | |
| | Perfume | 0.5 |
| | TOTAL: | 100 |

1) Ucare Polymer JR-400, Noveon
2) Schweizerhall
3) Glydant, Lonza
4) Texapon NSO IS, Cognis
5) Tego Betain F 50, Evonik
6) Amphotensid GB 2009, Zschimmer & Schwarz
7) Monomuls 90 L-12, Gruenau
8) Nipagin Monosodium, NIPA

### Example 6

### Antiperspirant Roll-On Emulsion Composition

Microcapsules of the present invention are weighed and mixed in antiperspirant roll-on emulsion composition to add the equivalent of 0.2% perfume.

TABLE 13
Antiperspirant roll-on emulsion composition

| Ingredient | Amount (wt %) |
|---|---|
| Steareth-21) (Part A) | 3.25 |
| Steareth-212) (Part A) | 0.75 |
| PPG-15 Stearyl Ether3) (Part A) | 4 |
| WATER deionised (Part B) | 51 |
| Aluminum Chlorohydrate 50% | 40 |
| aqueous solution4) (Part C) | |
| Fragrance (Part D) | 1 |

1) BRIJ 72; origin : ICI
2) BRIJ 721; origin : ICI
3) ARLAMOL E; origin : UNIQEMA-CRODA
4) LOCRON L; origin : CLARIAN

Part A and B are heated separately to 75° C.; Part A is added to Part B under stirring and the mixture is homogenized for 10 min. Then, the mixture is cooled under stirring; and Part C is slowly added when the mixture reached 45° C. and Part D when the mixture reached at 35° C. while stirring. Then the mixture is cooled to room temperature.

### Example 7

### Deodorant Spray Composition

Microcapsules of the present invention are weighed and mixed in antiperspirant roll-on emulsion composition to add the equivalent of 0.2% perfume.

TABLE 14
Deodorant spray composition

| Ingredient | Amount (wt %) |
|---|---|
| Ethanol 95 % | 90.65 |
| Triclosan1) | 0.26 |
| Isopropyl miristate | 9.09 |

1) Irgasan®DP 300;
trademark and origin : BASF

All the ingredients according to the sequence of Table 11 are mixed and dissolved. Then the aerosol cans are filled, crimp and the propellant is added (Aerosol filling: 40% active solution 60% Propane/Butane 2.5 bar).

### Example 8

### Shower-Gel Composition

Microcapsules of the present invention are weighed and mixed in the following composition to add the equivalent of 0.2% perfume.

TABLE 15
Shower gel composition

| | Amount | |
|---|---|---|
| Ingredients | (% wt) | Function |
| WATER deionized | 49.350 | Solvent |
| Tetrasodium EDTA ¹⁾ | 0.050 | Chelating |
| | | agent |
| Acrylates Copolymer²⁾ | 6.000 | Thickener |
| Sodium C12-C15 | 35.000 | Surfactant |
| Pareth Sulfate ³⁾ | | |
| Sodium Hydroxide 20% | 1.000 | pH adjuster |
| aqueous solution | | |
| Cocamidopropyl Betaine⁴⁾ | 8.000 | Surfactant |
| Methylchloroisothiazolinone | 0.100 | Preservative |
| and Methylisothiazolinone⁵⁾ | | |
| Citric Acid (40%) | 0.500 | pH adjuster |

2) EDETA B POWDER; trademark and origin: BASF
3) CARBOPOL AQUA SF-1 POLYMER; trademark and origin: NOVEON
4) ZETESOL AO 328 U; trademark and origin: ZSCHIMMER & SCHWARZ
5) TEGO-BETAIN F 50; trademark and origin: GOLDSCHMIDT
6) KATHON CG; trademark and origin: ROHM & HASS

### Example 9

### Unit Dose Formulation

A sufficient amount of exemplified microcapsules is weighed and mixed in a unit dose formulation to add the equivalent of 0.2% perfume.
The unit dose formulation can be contained in a PVOH (polyvinyl alcohol) film.

TABLE 16
Unit dose composition

| | Concentration |
|---|---|
| Ingredients | [wt % ] |
| C12 - C14 alkyl poly ethoxylate | 15 |
| C12 - C14 alkyl poly ethoxylate | 9.5 |
| sulfate Mono Ethanol Amine | |
| salt | |
| Linear Alkylbenzene sulfonic acid | 17 |
| Citric Acid | 0.5 |
| C12 - C18 Fatty Acid | 17 |
| Enzymes | 1.2 |
| Fluorescent brightener | 0.3 |
| 1,2 propanediol | 12 |
| Glycerol | 9 |
| Sodium Hydroxide | 1 |
| Mono Ethanol Amine | 6 |
| PDMS | 2.5 |
| Potassium sulphite | 0.2 |
| water | 8.8 |
| Total | 100 |

1. A process for preparing a microcapsule slurry, comprising the steps of:
1) suspending coacervate particles in a solvent to form a dispersing phase;
2) admixing at least one polyfunctional monomer with a hydrophobic material to form an oil phase;
3) adding the oil phase to the dispersing phase and mixing them to form a two-phase Pickering emulsion under conditions allowing the formation of a microcapsule slurry by interfacial polymerization.

2. The process according to claim 1, wherein the coacervate particles are prepared by a flash nanoprecipitation process.

3. The process according to claim 1, wherein the coacervate particles comprise at least one polymer.

4. The process according to claim 1, wherein the coacervate particles comprise a first polymer and a second polymer.

5. The process according to claim 4, wherein the first polymer contains at least one cationic group.

6. The process according to according to claim 4, wherein the first polymer is selected from the group consisting of proteins, chitosan and derivatives, cationic modified polysaccharides, polyethyleneimine, poly(amidoamine)s, poly(amino-co-ester), cationic polyacrylate, and mixtures thereof.

7. The process according to claim 4, wherein the second polymer is selected from the group of polymers consisting of modified starch, gum arabic, chitosan, sodium caseinate, alginate salts, cellulose derivatives, guar gum, pectinate salts, pectin, carrageenan, polyacrylic and methacrylic acid, xanthan gum, polyanions selected from the group consisting of sodium triphosphate, trisodium trimetaphosphate, sodium pyrophosphate, and dextran sulfate sodium salt, and mixtures thereof.

8. The process according to claim 1, wherein the coacervate particle comprises a polymer and an oppositely charged poly ion with the polymer.

9. The process according to claim 1, wherein the coacervate particles comprise an active ingredient, preferably chosen in the group consisting of a cosmetic ingredient, skin caring ingredient.

10. The process according to claim 1, wherein the polyfunctional monomer is selected from the group consisting of at least one isocyanate, anhydride or maleic anhydride, acyl chloride, epoxide, (meth) acrylate monomers, alkoxysilane, and mixtures thereof.

11. A microcapsule slurry obtained by the process according to claim 1, wherein the microcapsules comprise:
a core comprising a hydrophobic material, and
a composite shell comprising a first material and a second material, wherein:
the first material and the second material are different,
the first material is coacervate particles, and
the second material is a polymeric material.

12. The microcapsule slurry according to claim 11, wherein the weight ratio in the slurry between the first material and the second material is between 10:90 and 99:1.

13. The microcapsule slurry according to claim 11, wherein the second material is present in an amount less than 5%, by weight based on the total weight of the microcapsule slurry.

14. A consumer product comprising:
a personal care active base, and
the microcapsule slurry according to claim 11,
wherein the consumer product is in the form of a personal care composition.

15. A consumer product comprising:
a home care or a fabric care active base, and
the microcapsule slurry according to claim 11,
wherein the consumer product is in the form of a home care or a fabric care composition.

16. The process according to claim 4, wherein the first polymer contains at least one cationic group selected from the group consisting of amino groups, azole groups, amide groups, quaternary ammonium cations, and guanidinium cations.

17. The process according to claim 1, wherein the active ingredient is selected from the group consisting of a cosmetic ingredient, skin caring ingredient, perfume ingredient, flavor ingredient, malodour counteracting ingredient, antimicrobial ingredient, pharmaceutical or agrochemical ingredient, a sanitizing ingredient, an insect repellent or attractant, and mixtures thereof.

18. The microcapsule slurry according to claim 11, wherein the core is an oil-based core.

19. The microcapsule slurry according to claim 11, wherein the hydrophobic material is a perfume oil.

20. The microcapsule slurry according to claim 11, wherein the weight ratio in the slurry between the first material and the second material is between 30:70 and 99:1.
